(19) United States
(12) Patent Application Publication
(10) Pub. No.: US 2025/0134445 A1
(43) Pub. Date: May 1, 2025

(54) APPARATUSES FOR PREDICTING AND USING OLFACTORY PROFILES

(71) Applicant: Sony Group Corporation, Tokyo (JP)

(72) Inventors: Tarek Richard BESOLD, Basingstoke (GB); Priyadarshini KUMARI, Basingstoke (GB); Gao PEI, Basingstoke (GB); Daniel SHIN, Basingstoke (GB)

(73) Assignee: Sony Group Corporation, Tokyo (JP)

(21) Appl. No.: 18/494,546

(22) Filed: Oct. 25, 2023

(51) Int. Cl. A61B 5/00

(52) U.S. Cl. CPC A61B 5/4011; A61B 5/7264

(57) ABSTRACT

Aspects of the present disclosure relate to an apparatus for predicting an olfactory profile of a molecule, the apparatus comprising memory circuitry, machine-readable instructions, and processor circuitry to execute the machine-readable instructions to obtain a first representation of the molecule and a second representation of the molecule, process the first representation using at least one first machine-learning model to obtain a first predicted olfactory profile of the molecule, process the second representation using at least one second machine-learning model to obtain a second predicted olfactory profile of the molecule, process the first predicted olfactory profile and the second predicted olfactory profile, or a combined version of the first predicted olfactory profile and the second predicted olfactory profile, using a third machine-learning model, the third machine-learning model being trained to output a third predicted olfactory profile of the molecule.

### FIELD

Examples relate to apparatuses for predicting and using olfactory profiles, and for training corresponding machine-learning models.

### BACKGROUND

For humans and other animals, the sense of smell provides crucial information in many situations of everyday life. Still, the study of olfactory perception has received only limited attention outside of the biological sciences. From an AI (Artificial Intelligence) perspective, the complexity of the interactions between olfactory receptors and volatile molecules and the scarcity of comprehensive olfactory datasets, present unique challenges in this sensory domain. Previous works in academia have explored the relationship between molecular structure and odor descriptors using fully supervised training approaches. However, these methods are data-intensive and poorly generalize due to labeled data scarcity, particularly for rare-class samples.

### SUMMARY

Various examples of the present disclosure are based on the finding, that commonly used representations of molecules, such as textual representations or graph representations of molecules, often only represent certain aspects of the respective molecules, depending on the focus of the respective representation. In effect, each of these representations, taken alone, only provides an incomplete representation of the respective molecule. By using multiple representations of molecules (e.g., textual, and graph-based representations) as a starting point for the prediction of an olfactory profile of a molecule by a first and second machine-learning model, a more complete composite representation of the respective molecule may be used as the basis. Moreover, by training a third machine-learning model to combine the predictions of two models being based on two different representations, a prediction of an olfactory profile with a higher prediction accuracy can be achieved. To further improve the prediction accuracy, and to overcome the challenge of labelled data scarcity, the first and second machine-learning model may each be trained on a subset of the labels each, such that the full benefit of the combination technique can be leveraged. Finally, to leverage the qualities of existing machine-learning models, the first and second machine-learning model may each be derived from machine-learning models being trained on another purpose, such a sequence-to-sequence transformation.

Some aspects of the present disclosure relate to an apparatus for predicting an olfactory profile of a molecule. The apparatus comprises memory circuitry, machine-readable instructions, and processor circuitry to execute the machine-readable instructions to obtain a first representation of the molecule and a second representation of the molecule. The processor circuitry is to process the first representation using at least one first machine-learning model to obtain a first predicted olfactory profile of the molecule. The processor circuitry is to process the second representation using at least one second machine-learning model to obtain a second predicted olfactory profile of the molecule. The processor circuitry is to process the first predicted olfactory profile and the second predicted olfactory profile, or a combined version of the first predicted olfactory profile and the second predicted olfactory profile, using a third machine-learning model, the third machine-learning model being trained to output a third predicted olfactory profile of the molecule. By using multiple representations of molecules (e.g., textual, and graph-based representations) as a starting point for the prediction of an olfactory profile of a molecule by a first and second machine-learning model, a more complete composite representation of the respective molecule may be used as the basis. Moreover, by using a trained third machine-learning model to combine the predictions of two models being based on two different representations, a prediction of an olfactory profile with a higher prediction accuracy can be achieved.

For example, as outlined above, at least the third predicted olfactory profile may represent a plurality of olfactory labels. At least a component of the at least one first machine-learning model may be trained to predict a first subset of the plurality of olfactory labels and at least a component of the at least one second machine-learning model may be trained to predict a second subset of the plurality of olfactory labels (with the first subset being disjoint from the second subset). This may further improve the prediction accuracy, in particular with respect to scarcely represented olfactory labels, as the full benefit of the combination technique can be leveraged.

For example, the third machine-learning model may be trained to generate the third predicted olfactory profile based on the labels predicted by the at least one first machine-learning model and the at least one second machine-learning model. In particular, the third machine-learning model may be trained to advantageously combine the results provided by the first and second machine-learning model.

In some cases, the third machine-learning model may be provided with both the first and the second predicted olfactory profile separately. This increases the amount of data to be processed by the third machine-learning model, which may increase the time required for training the third machine-learning model. Alternatively, the processor circuitry may combine the first predicted olfactory profile and the second predicted olfactory profile to generate an input to the third machine-learning model. This way, the third machine-learning model has to process fewer inputs, and the respective predictions of the first machine-learning model may be inherently combined in the input provided to the third machine-learning model, which may facilitate the training of the third machine-learning model.

For example, the first predicted olfactory profile and the second predicted olfactory profile may be combined using one of concatenation, element-wise summation, and multiplication. Experiments have shown that these types of combination are particularly useful, with a slight additional performance advantage for concatenation and element-wise multiplication.

In various examples, the at least one first machine-learning model and the at least one first machine-learning model each comprise a pre-trained machine-learning model to generate an embedding of the molecule and a predictor machine-learning model to predict the respective first and second predicted olfactory profile based on the respective embedding of the molecule. This may leverage the qualities of existing machine-learning models and thus reduce the effort for training the machine-learning pipeline.

For example, the pre-trained machine-learning model may be trained using self-supervised training. Self-supervised techniques are useful, as they require no or little labeled data and can thus be applied onto the vast corpus of molecules with little manual intervention. Experiments have shown that models trained using self-supervised techniques can provide embeddings that are highly suitable for the downstream olfactory prediction task.

In various examples, at least one of the pre-trained machine-learning models may be a model for generating an embedding or representation of the molecule based on a graph representation of the molecule. Graph representations may be useful for representing the structure of the respective molecules, as molecular graphs are more effective in capturing key elements for modeling olfactory perception (such as the presence or absence of atoms, types of atomic bonds, orientation, and topology).

In various examples, at least one of the pre-trained machine-learning models may be a model for generating an embedding or representation of the molecule based on a textual representation of the molecule. This may improve the availability of suitable pre-trained machine-learning models, as many suitable models exist.

In various examples, the third machine-learning model and the predictor machine-learning models are trained together using end-to-end training. This may facilitate training, as only the different representations of the molecule and the ground truth olfactory profile of the molecule are required.

To yield a more complete representation of the respective molecule, the first representation of the molecule may be according to a first modality, and the second representation of the molecule may be according to a second modality being different from the first modality. For example, at least one of the first modality and the second modality may be one of a textual representation of the molecule, a graph representation of the molecule, an image representation of the molecule and a multi-dimensional embedding of the molecule. The different modalities may represent different aspects of the respective molecules, leading to a more complete representation of the molecule.

For example, the processor circuitry may obtain at least one further representation of the molecule, process the at least one further representation using at least one further machine-learning model to obtain at least one further predicted olfactory profile of the molecule, and process the first, second and at least one further predicted olfactory profile, or a combined version of the first, second and at least one further predicted olfactory profile using the third machine-learning model to generate the third predicted olfactory profile. By using additional representations of the molecule, a more complete composite representation can be achieved, which can further benefit the prediction accuracy.

In some examples, the processor circuitry may obtain a plurality of representations for a plurality of molecules, process the plurality of representations to obtain a plurality of third predicted olfactory profiles, and store the plurality of third olfactory profiles together with information on the respective molecule in a data structure. This way, a library or database of molecules can be generated, which can be used to select molecules having a desired olfactory profile.

Accordingly, the processor circuitry may select one or more molecules from the data structure based on a desired olfactory profile. This way, suitable molecules may be selected based on a desired scent (i.e., olfactory profile).

For example, the third olfactory profile may be provided for the purpose of selecting the molecule for use in one of a perfume, perfume component for another substance, cosmetic substance, and food item. In other words, the present techniques may be used in product development, helping engineers and researchers to select suitable molecules. For example, selected scents and corresponding molecules may be used by recipe designers to identify complementary foodstuffS and quantities and proportions thereof to include in a recipe, ensuring that scents and/or flavours combined together are pleasing. Output calculations of the present disclosure, such as the third predicted olfactory profile, may be applied in a robotic dosing device which selects from available molecules or ingredients in calculated proportions to fabricate or assemble a recipe. For example, the predicted third olfactory profile of the molecule can enable development of generative machine learning model for synthesizing novel molecule(s) with a specific odor profile.

In various examples, the processor circuitry includes at least one of a central processing unit, a graphics processing unit, an artificial intelligence accelerator, a field-programmable gate array, and an application-specific integrated circuit. These types of processor circuitry are particularly suited for inference tasks.

Some aspects of the present disclosure relate to an apparatus for selecting a molecule, the apparatus comprising memory circuitry, machine-readable instructions, and processor circuitry to execute the machine-readable instructions to select one or more molecules from a data structure based on a desired olfactory profile, with the data structure being generated by the above apparatus for predicting an olfactory profile of a molecule, and provide information on the one or more molecules for the purpose of selecting the one or more molecules for use in one of a perfume, perfume component for another substance, cosmetic substance, and food item. This way, suitable molecules may be selected based on a desired scent (i.e., olfactory profile). In effect, the present techniques may be used in product development, helping engineers and researchers to select suitable molecules.

Some aspects of the present disclosure relate to an apparatus for training machine-learning models. The apparatus comprises memory circuitry, machine-readable instructions, and processor circuitry to execute the machine-readable instructions to obtain training data. The training data comprises information on a plurality of molecules and associated olfactory profiles of the plurality of molecules. The processor circuitry is to train at least a component of at least one first machine-learning model, at least a component of at least one second machine-learning model, and a third machine-learning model using the training data. The at least one first machine-learning model is trained to output a first predicted olfactory profile of a molecule based on a first representation of the molecule. The at least one second machine-learning model is trained to output a second predicted olfactory profile of the molecule based on a second representation of the molecule. The third machine-learning model is trained to output a third predicted olfactory profile of the molecule using the first predicted olfactory profile and the second predicted olfactory profile, or a combined version of the first predicted olfactory profile and the second predicted olfactory profile, as input. This way, the machine-learning models used by the apparatus for predicting an olfactory profile of a molecule can be trained.

According to an example, at least a component of the at least one first machine-learning model, at least a component of the at least one second machine-learning model, and the third machine-learning model are trained using supervised learning. Supervised learning is possible in this case, as a ground truth with labels is available for training the respective models.

In various examples, at least the third predicted olfactory profile represents a plurality of olfactory labels. At least a component of the at least one first machine-learning model may be trained to predict a first subset of the plurality of olfactory labels and at least a component of the at least one second machine-learning model may be trained to predict a second subset of the plurality of olfactory labels. This may further improve the prediction accuracy, in particular with respect to scarcely represented olfactory labels, as the full benefit of the combination technique can be leveraged.

According to an example, the at least one first machine-learning model and the at least one first machine-learning model each comprise a pre-trained machine-learning model to generate an embedding of the molecule and a predictor machine-learning model to predict the respective first and second predicted olfactory profile based on the respective embedding of the molecule. The third machine-learning model and the predictor machine-learning models may be trained using the training data. This may leverage the qualities of existing machine-learning models and thus reduce the effort for training the machine-learning pipeline.

According to an example, the pre-trained machine-learning models remain unmodified when the third machine-learning model and the predictor machine-learning models are trained using the training data. This way, the effort required for training may be reduced.

In various examples, the third machine-learning model and the predictor machine-learning models are trained together, using the training data, using end-to-end training. This may further improve the prediction accuracy.

### BRIEF DESCRIPTION OF THE FIGURES

Some examples of apparatuses and/or methods will be described in the following by way of example only, and with reference to the accompanying figures, in which:

FIG. 1a shows a schematic diagram of an example of an apparatus for predicting an olfactory profile of a molecule, and of a computer system comprising such an apparatus;

FIG. 1b shows a flow chart of an example of a method for predicting an olfactory profile of a molecule;

FIG. 2a shows a schematic diagram of an example of an apparatus for selecting a molecule, and of a computer system comprising the apparatus;

FIG. 2b shows a flow chart of an example of a method for selecting a molecule;

FIG. 3a shows a schematic diagram of an example of an apparatus for training machine-learning models, and of a computer system comprising the apparatus;

FIG. 3b shows a flow chart of an example of a method for training machine-learning models;

FIG. 4 shows a diagram of a distribution of perceptual descriptors of odorants on an entire dataset;

FIG. 5 shows a schematic diagram of a SMILES (simplified molecular-input line-entry system) transformer trained through the self-supervised SMILES-IUPAC (International Union of Pure and Applied Chemistry) translation task;

FIG. 6 shows a schematic diagram of the MolCLR framework optimized for olfactory perception task;

FIG. 7 shows a schematic diagram of a label balancer technique;

FIGS. 8a and 8b show diagrams of the performance of perceptual features learned with and without transfer learning;

FIG. 9 shows a diagram of an ablation study to show the benefit of transfer learning on uni-modal and multi-modal representations;

FIG. 10 shows a diagram of a distribution of test samples where both SMILES and graph models make (dis)similar predictions;

FIG. 11 shows a diagram of a performance comparison between classical multi-modal fusion technique (Multi-Layer Perceptron, MLP, head) and label balancer;

FIG. 12 shows a diagram showing a performance gain by the label balancer over the MLP head approach on most-dense to most-sparse classes;

FIG. 13 shows a visualization of a molecular representation learned by the proposed model via t-SNE (t-distributed stochastic neighbor embedding);

FIG. 14 show the AUROC (Area Under the Receiver Operating Characteristic) of the MLP head fusion approach and of the label balancer approach; and

FIG. 15 shows a table of performance comparisons of label balancer and MLP head on each class.

### DETAILED DESCRIPTION

Some examples are now described in more detail with reference to the enclosed figures. However, other possible examples are not limited to the features of these embodiments described in detail. Other examples may include modifications of the features as well as equivalents and alternatives to the features. Furthermore, the terminology used herein to describe certain examples should not be restrictive of further possible examples.

Throughout the description of the figures same or similar reference numerals refer to same or similar elements and/or features, which may be identical or implemented in a modified form while providing the same or a similar function. The thickness of lines, layers and/or areas in the figures may also be exaggerated for clarification.

When two elements A and B are combined using an “or”, this is to be understood as disclosing all possible combinations, i.e., only A, only B as well as A and B, unless expressly defined otherwise in the individual case. As an alternative wording for the same combinations, “at least one of A and B” or “A and/or B” may be used. This applies equivalently to combinations of more than two elements.

If a singular form, such as “a”, “an” and “the” is used and the use of only a single element is not defined as mandatory either explicitly or implicitly, further examples may also use several elements to implement the same function. If a function is described below as implemented using multiple elements, further examples may implement the same function using a single element or a single processing entity. It is further understood that the terms “include”, “including”, “comprise” and/or “comprising”, when used, describe the presence of the specified features, integers, steps, operations, processes, elements, components and/or a group thereof, but do not exclude the presence or addition of one or more other features, integers, steps, operations, processes, elements, components and/or a group thereof.

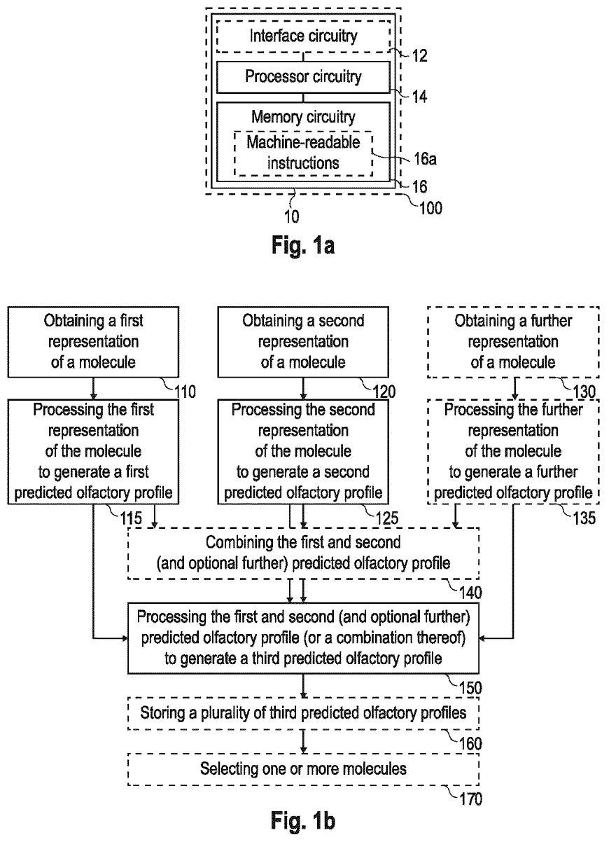

FIG. 1a shows a schematic diagram of an example of an apparatus 10 for predicting an olfactory profile of a molecule, and of a computer system 100 comprising such an apparatus 10. The apparatus 10 comprises circuitry to provide the functionality of the apparatus 10. For example, the circuitry of the apparatus 10 may be configured to provide the functionality of the apparatus 10. For example, the apparatus 10 of FIG. 1a comprises (optional) interface circuitry 12, processor circuitry 14, and memory/storage circuitry 16. For example, the processor circuitry 14 may be coupled with the interface circuitry 12 and/or with the memory/storage circuitry 16. For example, the processor circuitry 14 may provide the functionality of the apparatus, in conjunction with the interface circuitry 12 (for communicating with other entities inside or outside the computer system 100), and the memory/storage circuitry 16 (for storing information, such as machine-readable instructions and/or machine-learning models). In general, the functionality of the processor circuitry 14 may be implemented by the processor circuitry 14 executing machine-readable instructions. Accordingly, any feature ascribed to the processor circuitry 14 may be defined by one or more instructions of a plurality of machine-readable instructions. The apparatus 10 may comprise the machine-readable instructions 16a, e.g., within the memory or storage circuitry 16.

The processor circuitry 14 is to obtain a first representation of the molecule and a second representation of the molecule. The processor circuitry 14 is to process the first representation using at least one first machine-learning model to obtain a first predicted olfactory profile of the molecule. The processor circuitry 14 is to process the second representation using at least one second machine-learning model to obtain a second predicted olfactory profile of the molecule. The processor circuitry 14 is to process the first predicted olfactory profile and the second predicted olfactory profile, or a combined version of the first predicted olfactory profile and the second predicted olfactory profile, using a third machine-learning model, the third machine-learning model being trained to output a third predicted olfactory profile of the molecule. For example, the processor circuitry 14 may provide/output the output of the third machine-learning model, i.e., the third predicted olfactory profile.

FIG. 1b shows a flow chart of an example of a corresponding method for predicting an olfactory profile of a molecule. The method comprises obtaining 110, 120 the first representation of the molecule and the second representation of the molecule. The method comprises processing 115 the first representation using the at least one first machine-learning model to obtain the first predicted olfactory profile of the molecule. The method comprises processing 125 the second representation using the at least one second machine-learning model to obtain the second predicted olfactory profile of the molecule. The method comprises processing 150 the first predicted olfactory profile and the second predicted olfactory profile, or the combined version of the first predicted olfactory profile and the second predicted olfactory profile, using the third machine-learning model, the third machine-learning model being trained to output a third predicted olfactory profile of the molecule. For example, the method may be performed by a computer system, e.g., by processor circuitry of a computer system, such as the processor circuitry 14 of the computer system 100.

In the following, the features of the apparatus 10, the method and of a corresponding computer program will be introduced in more detail with reference to the apparatus 10. Features introduced in connection with the apparatus 10 may likewise be introduced into the corresponding method and computer program.

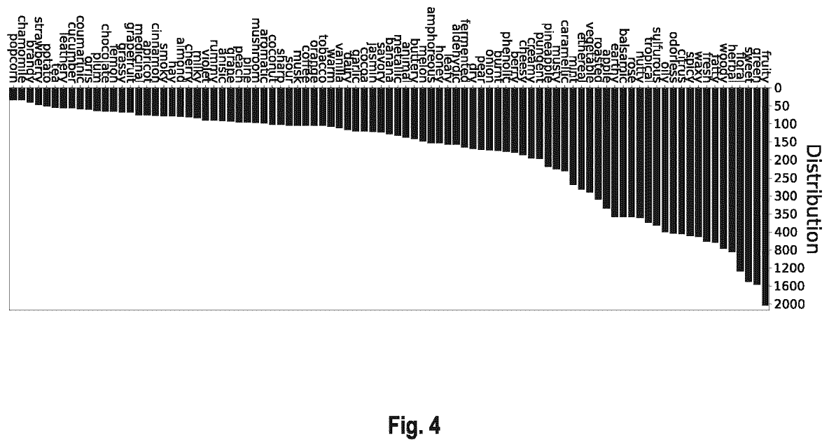

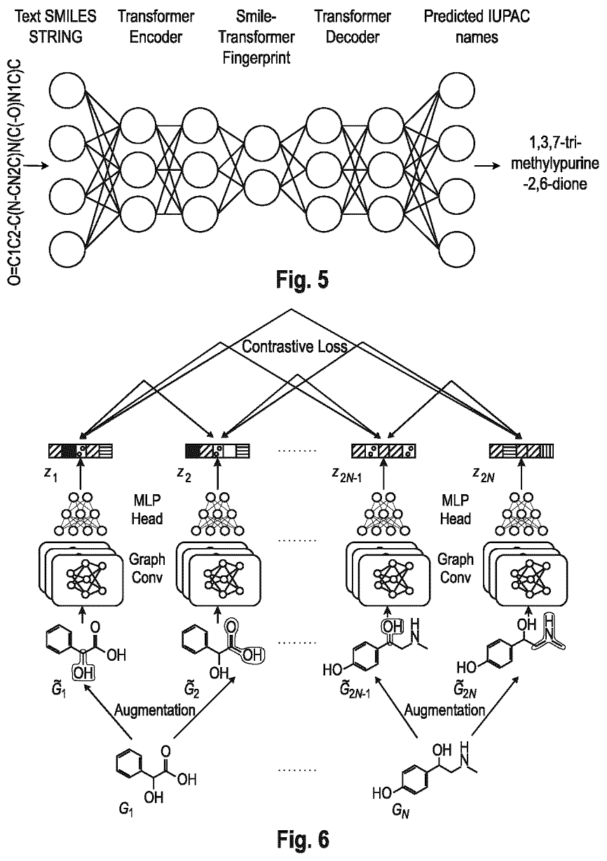

Various examples of the present disclosure are based on the finding, that commonly used representations of molecules, such as textual representations or graph representations of molecules, often only represent certain aspects of the respective molecules, depending on the focus of the respective representation. In effect, each of these representations, taken alone, only provides an incomplete representation of the respective molecule. By using multiple representations of molecules (e.g., textual, and graph-based representations) as a starting point for the prediction of an olfactory profile of a molecule by a first and second machine-learning model, a more complete composite representation of the respective molecule may be used as the basis. In the proposed concept, at least two different representations are used as a starting point—the first representation of the molecule, and the second representation of the molecule. These (at least) two representations are different from another. For example, the first and second representations may be different textual representations or sequence representations of the respective molecule, e.g., according to the SMILES (simplified molecular-input line-entry system) notation or according to the IUPAC (International Union of Pure and Applied Chemistry). Alternatively, the first and second representations may be different graph-based representations, or different image-based representations of the respective molecule. Preferably, however, the first and second (and further) representations may be based on different modalities. In other words, the first representation of the molecule may be according to a first modality, and the second representation of the molecule may be according to a second modality being different from the first modality. To give an example (also being used in connection with FIGS. 4 to 15, and in particular FIGS. 5 to 7, the first representation may be a textual representation of the molecule (e.g., according to the SMILES notation) and the second representation may be a graph-based representation of the molecule. More generally, however, the first and second representation may be based on different representations, such as a textual representation and a graph representation, a textual representation and an image representation, a graph representation, an image representation, a multi-dimensional representation/embedding (such as MORDRED) etc. In other words, the first modality and the second modality may be one representation/modality of the list of a textual representation of the molecule, a graph representation of the molecule, an image representation of the molecule and a multi-dimensional embedding of the molecule, and the second modality may be another representation/modality of the list of a textual representation of the molecule, a graph representation of the molecule, an image representation of the molecule and a multi-dimensional embedding of the molecule. In this context, a modality is a form of representation of the molecule, e.g., textual, image, graph, or multi-dimensional embedding.

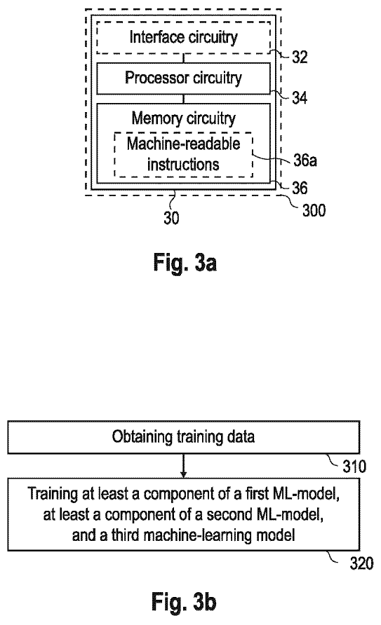

These different representations are then processed using the at least one first and the at least one second machine-learning model, respectively. In general terms, both the at least one first and at least one second machine-learning model serve the purpose of predicting the olfactory profile of the molecule. While this can be done from scratch, e.g., by training two different machine-learning models, using supervised learning, to predict the olfactory profile based on different representations, the computational effort being used for training the at least one first and at least one second machine-learning model may be reduced by re-using existing machine-learning models that are not necessarily being used for predicting olfactory profiles. For example, as outlined in connection with FIGS. 5 and 6, existing machine-learning models with other purposes, such as sequence-to-sequence translation (for translating between the SMILES and the IUPAC notation in the case of FIG. 5) and molecule property prediction (apart from olfactory prediction), such as the MolCLR framework/model discussed in connection with FIG. 6, may be used. Alternatively, or additionally, the MOLDRED model may be used, which is also discussed in connection with FIGS. 4 to 15. In other words, the at least one first machine-learning model and the at least one second machine-learning model may be based on, include, or be derived from pre-trained models that have been trained for a purpose other than olfactory prediction, e.g., using self-supervised training. In essence, transfer learning is applied on the results of the at least one first and at least one second machine-learning model. In the present case, they may be used to generate an embedding (i.e., a multi-dimensional representation) of the molecule, which may then be processed using one or more additional layers of a neural network (or using the existing layers, adapted to the olfactory prediction task) forming an MLP head to perform the olfactory prediction task. In more general terms, the at least one first machine-learning model and the at least one first machine-learning model may each comprise a pre-trained machine-learning model to generate an embedding of the molecule and a predictor machine-learning model to predict the respective first and second predicted olfactory profile based on the respective embedding of the molecule. In connection with FIGS. 4 to 15, the predictor machine-learning model is also denoted MLP (Multi-Layer Perceptron) head, which is the final layer or component of a MLP model, such as a neural network. The MLP head is responsible for making the final predictions or classifications based on the features or inputs provided to the model. The MLP head can be customized to suit different tasks or applications. The predictor machine-learning model, or MLP head, may be a layer of the pre-trained model being specifically adapted (i.e., trained) to the olfactory prediction task, or one or more additional layers being used to process an output of the pre-trained machine-learning models. More details on the training of the respective models will be given in connection with FIGS. 3a to 3c, and FIGS. 5, 6, and 7.

As the at least one first machine-learning model and the at least one second machine-learning model are used to process different representations, and preferably different representations being based on different modalities, different pre-trained machine-learning models being suitable for processing these representations/modalities may be used. For example, at least one of the pre-trained machine-learning models may be a model for generating an embedding or representation of the molecule based on a graph representation of the molecule. Similarly, at least one of the pre-trained machine-learning models may be a model for generating an embedding or representation of the molecule based on a textual representation of the molecule.

The third machine-learning model is then used to use the output provided by the at least one first machine-learning model and the at least one second machine-learning model to determine the third predicted olfactory profile, which is the desired output, i.e., the actual predicted olfactory profile being output. While the first, second and third predicted olfactory profile have similar names, the format of the three different olfactory profiles is not necessarily the same. As the first and second predicted olfactory profiles are provided by at least two different machine-learning models, in many cases, the first and second predicted olfactory profiles may have different formats, e.g., vectors having different numbers of entries/dimensions, and may also be different from the third predicted olfactory profile. In general, each of the first, second and third predicted olfactory profile may be an embedding, i.e., an n-dimensional vector, representing the respective predicted olfactory profile and molecule, with n potentially being different for each of the first, second and/or third predicted olfactory profile. In some cases, in particular when the first and second olfactory profile are combined before being input into the third machine-learning model, the number of dimensions of the first and second predicted olfactory profile may be reduced to a common number of dimensions, such that combinations, such as element-wise summation or element-wise multiplication/product are possible. In other words, the processor circuitry may reduce the vector dimensions of the first and/or second predicted olfactory profile to a common set of dimensions, and thus the same number of dimensions, before combining the first and second predicted olfactory profiles, and/or before inputting the first and second predicted olfactory profiles to the third machine-learning model.

As outlined above, in some cases, the first and second predicted olfactory profile may be combined prior to inputting into the third machine-learning model. In other words, the processor circuitry may combine the first predicted olfactory profile and the second predicted olfactory profile to generate an input to the third machine-learning model. Accordingly, as further shown in FIG. 1b, the method may comprise combining 140 the first predicted olfactory profile and the second predicted olfactory profile to generate an input to the third machine-learning model. In the evaluation section of the latter part of this document (FIGS. 4 to 15), a performance comparison on different combinations of predicted olfactory profiles based on SMILES, graph representations and MORDRED is included, which highlights advantages and disadvantages of different forms of combination. In general terms, the first predicted olfactory profile and the second predicted olfactory profile (and further predicted olfactory profile(s)) may be combined using one of concatenation, element-wise summation, and (element-wise) multiplication.

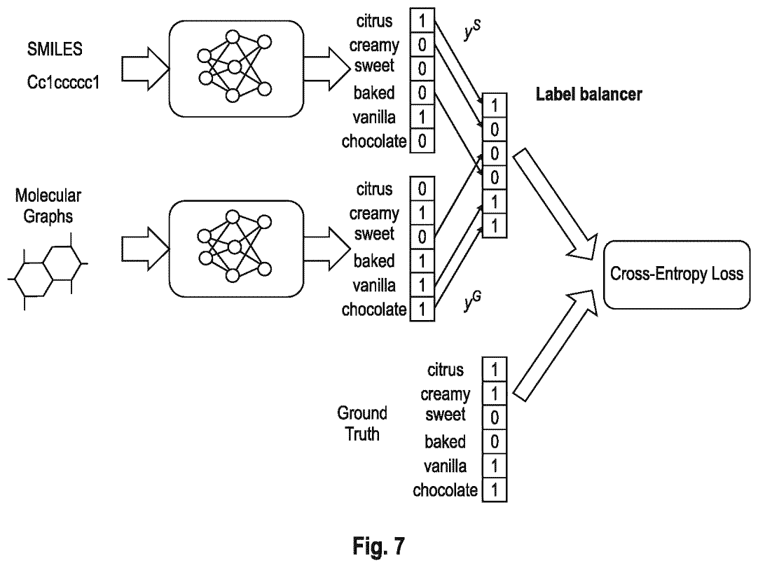

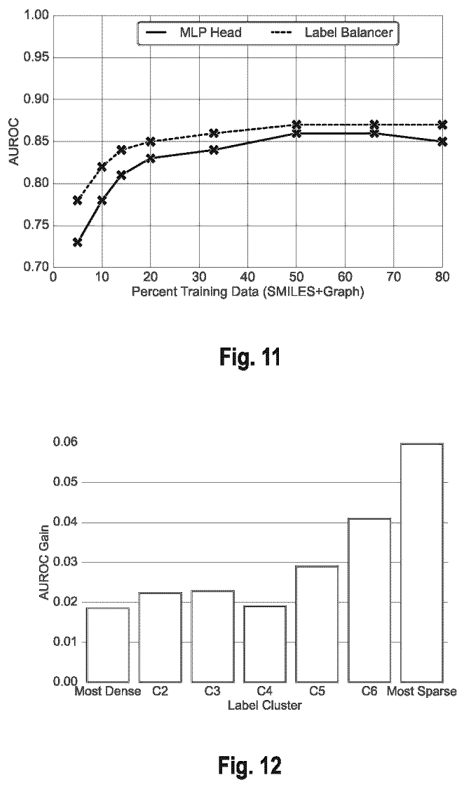

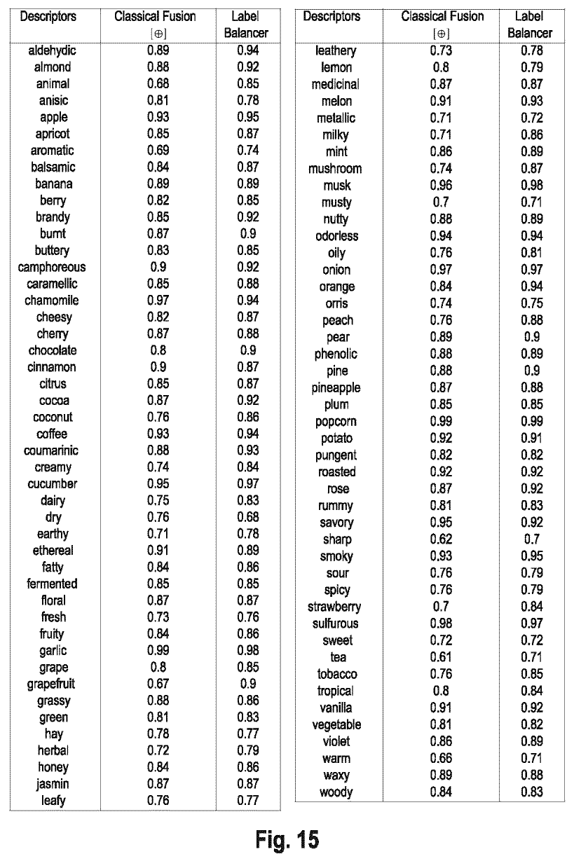

Core of the proposed concept is the third machine-learning model. In connection with FIGS. 4 to 15, and in particular FIG. 7, this third machine-learning model is denoted “label balancer”, as it balances the contributions of the first and second machine-learning model, improving the prediction quality of the combined pipeline relative to the respective outputs of the first and second machine-learning models, or deterministic combinations thereof (denoted “classical function”). An impact of the label balancer is shown in FIGS. 11 and 15, for example. In the proposed concept, the processor circuitry is to process the first predicted olfactory profile and the second predicted olfactory profile, or the combined version of the first predicted olfactory profile and the second predicted olfactory profile, using the third machine-learning model, the third machine-learning model being trained to output a third predicted olfactory profile of the molecule. Compared to other approaches, the results of the (at least) two machine-learning models are fed into another machine-learning model (the third machine-learning model/label balancer), which is trained to output the final predicted olfactory profile based on the results generated by the first and second machine-learning model. For this purpose, the third machine-learning model may be trained end-to-end with at least the olfactory predictor components of the at least one first and at least one second machine-learning model, so the prediction accuracy can be improved in an end-to-end manner. In other words, the third machine-learning model and the predictor machine-learning models (i.e., the MLP heads) may be trained together using end-to-end training.

To further improve prediction accuracy, and to address the challenge of rarely represented olfactory categories, another technique can be used. When combining the results of two different predictors, the prediction accuracy can be further improved if both of the machine-learning models providing the input for the third machine-learning model are specialized for only a portion of the space, such that the combination of the results by the third machine-learning model can yield a result that is based on the strengths of both the first and second machine-learning model. In the present case, this means that the different machine-learning models are trained to predict different subsets of olfactory labels. In more general terms, at least the third predicted olfactory profile may represent a plurality of olfactory labels. For example, for each label of the plurality of olfactory labels, the third predicted olfactory profile may comprise a binary classification (i.e., molecule exhibits smell according to the respective label or not) or a probability (i.e., probability that molecule exhibits smell according to the respective label on a real number scale from 0 to 1). For example, e.g., at each training iteration, at least a component of the at least one first machine-learning model (i.e., the predictor component) may be trained to predict a first subset of the plurality of olfactory labels and at least a component of the at least one second machine-learning model (i.e., the predictor component) may be trained to predict a second subset of the plurality of olfactory labels. For example, the first and second subset of labels may be disjoint, such that the component of the first and second machine-learning models are trained to predict different labels, at least on a per-iteration level. The third machine-learning model may then use the predicted labels from both machine-learning models. In other words, the third machine-learning model may be trained to generate the third predicted olfactory profile based on the labels predicted by the at least one first machine-learning model and the at least one second machine-learning model.

In general, the use of additional representations and/or modalities may be used to further improve the prediction result. For example, the processor circuitry may obtain at least one further representation of the molecule, process the at least one further representation using at least one further machine-learning model to obtain at least one further predicted olfactory profile of the molecule, and to process the first, second and at least one further predicted olfactory profile, or a combined version of the first, second and at least one further predicted olfactory profile using the third machine-learning model to generate the third predicted olfactory profile. Accordingly, as further shown in FIG. 1b, the method may comprise obtaining 130 at least one further representation of the molecule, processing 135 the at least one further representation using at least one further machine-learning model to obtain at least one further predicted olfactory profile of the molecule, and processing 150 the first, second and at least one further predicted olfactory profile, or a combined version of the first, second and at least one further predicted olfactory profile using the third machine-learning model to generate the third predicted olfactory profile.

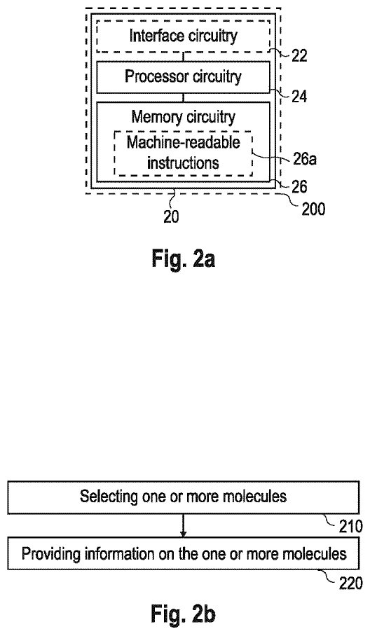

The prediction pipeline described herein may be used in various scenarios. In particular, it may be used during the development of new products. For example, the third olfactory profile may be provided for the purpose of selecting the molecule for use in one of a perfume, perfume component for another substance (e.g., a glue or lubricant), cosmetic substance, and food item. For this purpose, a library or database of olfactory profiles of molecules may be generated, which can be searched for a desired olfactory profile. For example, the processor circuitry may obtain a plurality of (first, second, and optional further) representations for a plurality of molecules, process the plurality of representations to obtain a plurality of third predicted olfactory profiles, and store (operation 160 of FIG. 1b) the plurality of third olfactory profiles together with information on the respective molecule in a data structure, such as a database or library. For example, the plurality of molecules, and corresponding representations, may be provided by a user, or obtained from another database, e.g., based on one or more further properties (such as toxicity, suitability as a lubricant/glue etc.) shared by the plurality of molecules. Someone developing a product may query the data structure to select one or more molecules, e.g., according to an olfactory profile/scent the user is looking for. For example, the processor circuitry may select one or more molecules from the data structure based on a desired olfactory profile. Accordingly, as further shown in FIG. 1b, the method may comprise selecting 170 one or more molecules from the data structure based on the desired olfactory profile. Subsequently, which is the focus of the apparatus, method, and computer program of FIGS. 2a and 2b, the processor circuitry may provide information on the one or more molecules for the purpose of selecting the one or more molecules for use in one of a perfume, perfume component for another substance, cosmetic substance, and food item, for a user looking to use the respective molecules as part of a product development process. For example, the information on the one or more molecules may comprise information on a chemical composition of the one or more molecules (e.g., the first, second and/or further representation) and information on the third predicted olfactory profile of the one or more molecules.

The interface circuitry 12 may correspond to one or more inputs and/or outputs for receiving and/or transmitting information, which may be in digital (bit) values according to a specified code, within a module, between modules or between modules of different entities. For example, the interface circuitry 12 may comprise circuitry configured to receive and/or transmit information.

For example, the processor circuitry 14 may be implemented using one or more processing units, one or more processing devices, any means for processing, such as a processor, a computer or a programmable hardware component being operable with accordingly adapted software. In other words, the described function of the processor circuitry 14 may as well be implemented in software, which is then executed on one or more programmable hardware components. Such hardware components may comprise a general-purpose processor, a Digital Signal Processor (DSP), a micro-controller, etc. For example, the processor circuitry 14 may include at least one of a central processing unit (CPU), a graphics processing unit (GPU), an artificial intelligence accelerator, a field-programmable gate array (FPGA), and an application-specific integrated circuit (ASIC).

For example, the memory or storage circuitry 16 may a volatile memory, e.g., random access memory, such as dynamic random-access memory (DRAM), and/or comprise at least one element of the group of a computer readable storage medium, such as a magnetic or optical storage medium, e.g., a hard disk drive, a flash memory, Floppy-Disk, Random Access Memory (RAM), Programmable Read Only Memory (PROM), Erasable Programmable Read Only Memory (EPROM), an Electronically Erasable Programmable Read Only Memory (EEPROM), or a network storage.

More details and aspects of the apparatus, method, and a corresponding computer program for predicting an olfactory profile of a molecule are mentioned in connection with the proposed concept or one or more examples described above or below (e.g., FIG. 2a to 15). The apparatus, method, and corresponding computer program for predicting an olfactory profile of a molecule may comprise one or more additional optional features corresponding to one or more aspects of the proposed concept, or one or more examples described above or below.

FIG. 2a shows a schematic diagram of an example of an apparatus 20 for selecting a molecule, and of a computer system 200 comprising the apparatus 20. The apparatus 20 comprises circuitry to provide the functionality of the apparatus 20. For example, the circuitry of the apparatus 20 may be configured to provide the functionality of the apparatus 20. For example, the apparatus 20 of FIG. 2a comprises (optional) interface circuitry 22, processor circuitry 24, and memory/storage circuitry 26. For example, the processor circuitry 24 may be coupled with the interface circuitry 22 and/or with the memory/storage circuitry 26. For example, the processor circuitry 24 may provide the functionality of the apparatus, in conjunction with the interface circuitry 22 (for communicating with other entities inside or outside the computer system 200), and the memory/storage circuitry 26 (for storing information, such as machine-readable instructions and/or machine-learning models). In general, the functionality of the processor circuitry 24 may be implemented by the processor circuitry 24 executing machine-readable instructions. Accordingly, any feature ascribed to the processor circuitry 24 may be defined by one or more instructions of a plurality of machine-readable instructions. The apparatus 20 may comprise the machine-readable instructions 26a, e.g., within the memory or storage circuitry 26.

The processor circuitry 24 is to select one or more molecules from a data structure based on a desired olfactory profile, with the data structure being generated by the apparatus of FIG. 1a. The processor circuitry 24 is to provide information on the one or more molecules for the purpose of selecting the one or more molecules for use in one of a perfume, perfume component for another substance, cosmetic substance, and food item.

FIG. 2b shows a flow chart of an example of a corresponding method for selecting a molecule. The method comprises selecting 210 one or more molecules from a data structure based on a desired olfactory profile, with the data structure being generated by the method of FIG. 1b. The method comprises providing 220 the information on the one or more molecules for the purpose of selecting the one or more molecules for use in one of a perfume, perfume component for another substance, cosmetic substance, and food item. For example, the method may be performed by a computer system, e.g., by processor circuitry of a computer system, such as the processor circuitry 24 of the computer system 200.

Selection of the one or more molecules, composition of the data structure, and provisioning of the information one the one or more molecules have been discussed in connection with FIG. 1a to 1b.

The interface circuitry 22 may correspond to one or more inputs and/or outputs for receiving and/or transmitting information, which may be in digital (bit) values according to a specified code, within a module, between modules or between modules of different entities. For example, the interface circuitry 22 may comprise circuitry configured to receive and/or transmit information.

For example, the processor circuitry 24 may be implemented using one or more processing units, one or more processing devices, any means for processing, such as a processor, a computer or a programmable hardware component being operable with accordingly adapted software. In other words, the described function of the processor circuitry 24 may as well be implemented in software, which is then executed on one or more programmable hardware components. Such hardware components may comprise a general-purpose processor, a Digital Signal Processor (DSP), a micro-controller, etc. For example, the processor circuitry 24 may include at least one of a central processing unit (CPU), a graphics processing unit (GPU), an artificial intelligence accelerator, a field-programmable gate array (FPGA), and an application-specific integrated circuit (ASIC).

For example, the memory or storage circuitry 26 may a volatile memory, e.g., random access memory, such as dynamic random-access memory (DRAM), and/or comprise at least one element of the group of a computer readable storage medium, such as a magnetic or optical storage medium, e.g., a hard disk drive, a flash memory, Floppy-Disk, Random Access Memory (RAM), Programmable Read Only Memory (PROM), Erasable Programmable Read Only Memory (EPROM), an Electronically Erasable Programmable Read Only Memory (EEPROM), or a network storage.

More details and aspects of the apparatus, method, and a corresponding computer program for selecting a molecule are mentioned in connection with the proposed concept or one or more examples described above or below (e.g., FIG. 1a to 1b, 3a to 15). The apparatus, method, and corresponding computer program for selecting a molecule may comprise one or more additional optional features corresponding to one or more aspects of the proposed concept, or one or more examples described above or below.

FIG. 3a shows a schematic diagram of an example of an apparatus 30 for training machine-learning models, and of a computer system 300 comprising the apparatus 30. The apparatus comprises circuitry to provide the functionality of the apparatus 30. For example, the circuitry of the apparatus 30 may be configured to provide the functionality of the apparatus 30. For example, the apparatus 30 of FIG. 3a comprises (optional) interface circuitry 32, processor circuitry 34, and memory/storage circuitry 36. For example, the processor circuitry 34 may be coupled with the interface circuitry 32 and/or with the memory/storage circuitry 36. For example, the processor circuitry 34 may provide the functionality of the apparatus, in conjunction with the interface circuitry 32 (for communicating with other entities inside or outside the computer system 300), and the memory/storage circuitry 36 (for storing information, such as machine-readable instructions and/or machine-learning models). In general, the functionality of the processor circuitry 34 may be implemented by the processor circuitry 34 executing machine-readable instructions. Accordingly, any feature ascribed to the processor circuitry 34 may be defined by one or more instructions of a plurality of machine-readable instructions. The apparatus 30 may comprise the machine-readable instructions 36a, e.g., within the memory or storage circuitry 36.

The processor circuitry 34 is to obtain training data. The training data comprises information on a plurality of molecules and associated olfactory profiles of the plurality of molecules. The processor circuitry 34 is to train at least a component of at least one first machine-learning model, at least a component of at least one second machine-learning model, and a third machine-learning model using the training data. The at least one first machine-learning model is trained to output a first predicted olfactory profile of a molecule based on a first representation of the molecule. The at least one second machine-learning model is trained to output a second predicted olfactory profile of the molecule based on a second representation of the molecule. The third machine-learning model is trained to output a third predicted olfactory profile of the molecule using the first predicted olfactory profile and the second predicted olfactory profile, or a combined version of the first predicted olfactory profile and the second predicted olfactory profile, as input.

FIG. 3b shows a flow chart of an example of a corresponding method for training machine-learning models. The method comprises obtaining 310 the training data. The method comprises training 320 at least a component of at least one first machine-learning model, at least a component of at least one second machine-learning model, and a third machine-learning model using the training data. For example, the method may be performed by a computer system, e.g., by processor circuitry of a computer system, such as the processor circuitry 34 of the computer system 300.

In the following, the features of the apparatus 30, the method and of a corresponding computer program will be introduced in more detail with reference to the apparatus 30. Features introduced in connection with the apparatus 30 may likewise be introduced into the corresponding method and computer program.

While FIGS. 1a to 2b relate to the application (i.e., inference) of the first, second and third machine-learning model, FIGS. 3a and 3b relate to their training. To illustrate the training, in the following, a brief introduction on machine-learning is given.

Machine learning refers to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and associated training content information, the machine-learning model “learns” to recognize the content of the images, so the content of images that are not included of the training images can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model.

Machine-learning models are trained using training input data. The examples specified above use a training method called “supervised learning”. In supervised learning, the machine-learning model is trained using a plurality of training samples, wherein each sample may comprise a plurality of input data values, and a plurality of desired output values, i.e., each training sample is associated with a desired output value. By specifying both training samples and desired output values, the machine-learning model “learns” which output value to provide based on an input sample that is similar to the samples provided during the training.

Apart from supervised learning, semi-supervised learning may be used. In semi-supervised learning, some of the training samples lack a corresponding desired output value. Supervised learning may be based on a supervised learning algorithm, e.g., a classification algorithm, a regression algorithm or a similarity learning algorithm. Classification algorithms may be used when the outputs are restricted to a limited set of values, i.e., the input is classified to one of the limited set of values. Regression algorithms may be used when the outputs may have any numerical value (within a range). Similarity learning algorithms are similar to both classification and regression algorithms but are based on learning from examples using a similarity function that measures how similar or related two objects are.

Apart from supervised or semi-supervised learning, unsupervised learning may be used to train the machine-learning model(s). In unsupervised learning, (only) input data might be supplied, and an unsupervised learning algorithm may be used to find structure in the input data, e.g., by grouping or clustering the input data, finding commonalities in the data. Clustering is the assignment of input data comprising a plurality of input values into subsets (clusters) so that input values within the same cluster are similar according to one or more (pre-defined) similarity criteria, while being dissimilar to input values that are included in other clusters.

Reinforcement learning is a third group of machine-learning algorithms. In other words, reinforcement learning may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such, that the cumulative reward is increased, leading to software agents that become better at the task they are given (as evidenced by increasing rewards).

Machine-learning algorithms are usually based on a machine-learning model. In other words, the term “machine-learning algorithm” may denote a set of instructions that may be used to create, train, or use a machine-learning model. The term “machine-learning model” may denote a data structure and/or set of rules that represents the learned knowledge, e.g., based on the training performed by the machine-learning algorithm. In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model(s), such as the first to third machine-learning models, may be an artificial neural network (ANN). ANNs are systems that are inspired by biological neural networks, such as can be found in a brain. ANNs comprise a plurality of interconnected nodes and a plurality of connections, so-called edges, between the nodes. There are usually three types of nodes, input nodes that receiving input values, hidden nodes that are (only) connected to other nodes, and output nodes that provide output values. Each node may represent an artificial neuron. Each edge may transmit information, from one node to another. The output of a node may be defined as a (non-linear) function of the sum of its inputs. The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e., to achieve a desired output for a given input. In at least some embodiments, the machine-learning model may be deep neural network, e.g., a neural network comprising one or more layers of hidden nodes (i.e., hidden layers), preferably a plurality of layers of hidden nodes.

In the present disclosure, three machine-learning models are distinguished—at least one first and at least one second machine-learning model that are used to predict an olfactory profile from a representation of a molecule, and a third machine-learning model that is used to combine the predictions of the first and second machine-learning model.

In many implementations, as outlined in connection with FIGS. 1a to 1b, and 5 to 7, the at least one first and at least one second machine-learning models may be based on, derived from, or include pre-trained models that have been trained for a different purpose, e.g., for sequence-to-sequence translation between representation etc. In particular, a pre-trained model may be used to generate an embedding of the respective representation being processed by the respective machine-learning model. For example, the at least one first machine-learning model and the at least one first machine-learning model may each comprise a pre-trained machine-learning model to generate an embedding of the molecule and a predictor machine-learning model to predict the respective first and second predicted olfactory profile based on the respective embedding of the molecule. Most if not all layers of these pre-trained machine-learning models may remain unchanged during the training process discussed herein, with only the predictor (which may be the MLP head of the pre-trained model, or another layer being used to process the embedding generated by the pre-trained model) being trained using the training data. In other words, the pre-trained machine-learning models may remain unmodified when the third machine-learning model and the predictor machine-learning models are trained using the training data.

What is being trained in the present case is thus a) the third machine-learning model, and b) the predictor machine-learning models (i.e., the MLP heads) of the at least one first and at least one second machine-learning model. These models may be trained together, in an end-to-end fashion, using supervised learning. In other words, the third machine-learning model and the predictor machine-learning models are trained using the training data, with the third machine-learning model and the predictor machine-learning models being trained together using end-to-end training.

To train the models or model components, supervised learning may be used. As outlined above, in supervised learning, a machine-learning model is trained using a plurality of training samples, wherein each sample comprises a plurality of input data values, and a plurality of desired output values, i.e., each training sample is associated with a desired output value. By specifying both training samples and desired output values, the machine-learning model “learns” which output value to provide based on an input sample that is similar to the samples provided during the training.

In the present case, the training data comprises suitable training data. In particular, the training data comprises information on a plurality of molecules and associated olfactory profiles of the plurality of molecules. For example, the training data may comprise, for each molecule of the plurality of molecules, at least two different representations of the molecule (to be input into the at least one first and at least one second machine-learning model), and the desired result, i.e., the associated olfactory profile, in a format that is identical to, or similar to, the third predicted olfactory profile. When performing end-to-end training, a first representation of the molecule may be input into the at least one first machine-learning model (e.g., the pre-trained machine-learning model thereof), and a second representation of the of the molecule may be input into the at least one second machine-learning model (e.g., the pre-trained machine-learning model thereof), as training input data. The pre-trained machine-learning models of the at least one first and at least one second machine-learning models may generate embeddings of the molecules, which are then used by the predictor machine-learning models (which may be part of the pre-trained models) to generate the predicted first and second predicted olfactory profiles. These predicted olfactory profiles may then be provided (e.g., in combined form or separately, and/or after the number of dimensions of the respective vectors has been reduced to match) as input to the third machine-learning model. The associated olfactory profile may be used as desired output of the third machine-learning model, with the predictor machine-learning models and the third machine-learning models being adjusted such that the predicted third machine-learning model becomes ever more similar to the desired output, i.e., the ground truth associated olfactory profile. For example, cross-entropy loss may be used for the supervised training.

In some examples, to further improve the performance of the prediction pipeline, the first and second machine-learning models may be trained to predict only subsets of the third olfactory profile. For example, at least the third predicted olfactory profile may represent a plurality of olfactory labels. At least a component of the at least one first machine-learning model (e.g., the predictor machine-learning model) may be trained to predict a first subset of the plurality of olfactory labels and at least a component of the at least one second machine-learning model (e.g., the predictor machine-learning model) may be trained to predict a second subset of the plurality of olfactory labels (with the first and second subset being disjoint). When this technique is employed, learning objectives are distributed across different modalities, enabling different models to optimize collaboratively for distinct subsets of labels. For example, in each training iteration, a random label division strategy may be used, where half of the labels are optimized using the at least one first machine-learning model, and the other half are optimized using the at least one second machine-learning model.

The interface circuitry 32 may correspond to one or more inputs and/or outputs for receiving and/or transmitting information, which may be in digital (bit) values according to a specified code, within a module, between modules or between modules of different entities. For example, the interface circuitry 32 may comprise circuitry configured to receive and/or transmit information.

For example, the processor circuitry 34 may be implemented using one or more processing units, one or more processing devices, any means for processing, such as a processor, a computer or a programmable hardware component being operable with accordingly adapted software. In other words, the described function of the processor circuitry 34 may as well be implemented in software, which is then executed on one or more programmable hardware components. Such hardware components may comprise a general-purpose processor, a Digital Signal Processor (DSP), a micro-controller, etc. For example, the processor circuitry 34 may include at least one of a central processing unit (CPU), a graphics processing unit (GPU), an artificial intelligence accelerator, a field-programmable gate array (FPGA), and an application-specific integrated circuit (ASIC).

For example, the memory or storage circuitry 36 may a volatile memory, e.g., random access memory, such as dynamic random-access memory (DRAM), and/or comprise at least one element of the group of a computer readable storage medium, such as a magnetic or optical storage medium, e.g., a hard disk drive, a flash memory, Floppy-Disk, Random Access Memory (RAM), Programmable Read Only Memory (PROM), Erasable Programmable Read Only Memory (EPROM), an Electronically Erasable Programmable Read Only Memory (EEPROM), or a network storage.

More details and aspects of the apparatus, method and a corresponding computer program for training machine-learning models are mentioned in connection with the proposed concept or one or more examples described above or below (e.g., FIG. 1a to 2b, 4 to 15). The apparatus, method and corresponding computer program for training machine-learning models may comprise one or more additional optional features corresponding to one or more aspects of the proposed concept, or one or more examples described above or below.

In the following, a concrete implementation example is given for the subject-matter discussed in connection with FIGS. 1a to 3b. The implementation example given in the following may be used, entirely or in part, to implement the apparatuses, methods and computer programs discussed in connection with FIGS. 1a to 3b.

Various examples of the present disclosure relate to a technique for improving or optimizing learning across multimodal transfer features for modeling olfactory perception.

The following examples may partially tackle the challenges of data scarcity and label skewness through multimodal transfer learning. The following discloses investigates the potential of large molecular foundation models trained on extensive unlabeled molecular data to effectively model olfactory perception. Additionally, the integration of different molecular representations, including molecular graphs and text-based SMILES encodings, to achieve data efficiency and generalization of the learned model, particularly on sparsely represented classes is explored. By leveraging complementary representations, an aim is to learn robust perceptual features of odorants. However, it is observed that traditional methods of combining modalities do not yield substantial gains in high-dimensional skewed label spaces. To address this challenge, a novel label-balancer technique is introduced that is specifically designed for high-dimensional multi-label and multi-modal training. The label-balancer technique distributes learning objectives across modalities to optimize collaboratively for distinct subsets of labels. Experimental results suggest that multi-modal transfer features learned using the label-balancer technique are more effective and robust, surpassing the capabilities of traditional uni- or multi-modal approaches, particularly on rare-class samples. The present disclosure relates to multimodal transfer learning, foundation models, perception modelling, and olfactory perception.

The human sense of smell playS a crucial role in many domains, including food and flavor perception, perfumery, assistive technology, healthcare, and increasingly also in multimodal user interface design. Despite its significance, olfactory perception has received relatively limited scientific attention outside of the biological sciences. This is largely due to several domain-specific challenges unique to this sensory domain, such as the complex interactions between hundreds of olfactory receptors and volatile molecules and the scarcity of comprehensive olfactory datasets.

While modeling olfactory perception is still in its early stages, machine learning has emerged as a promising approach for addressing various complex problems in a neighboring field, namely chemistry: drug discovery (Daria Grechishnikova. 2021. Transformer neural network for protein-specific de novo drug generation as a machine translation problem. Scientific reports 11, 1 (2021), 1-13) and protein folding (John Jumper, Richard Evans, Alexander Pritzel, Tim Green, Michael Figurnov, Olaf Ronneberger, Kathryn Tunyasuvunakool, Russ Bates, Augustin Židek, Anna Potapenko, et al. 2021. Highly accurate protein structure prediction with AlphaFold. Nature 596, 7873 (2021), 583-589) are only two of several examples. The enormous success of transformer and foundation models in the vision and the NLP domains, such as BERT (Jacob Devlin, Ming-Wei Chang, Kenton Lee, and Kristina Toutanova. 2018. Bert: Pre-training of deep bidirectional transformers for language understanding. arXiv preprint arXiv:1810.04805 (2018)), GPT (Alec Radford, Karthik Narasimhan, Tim Salimans, Ilya Sutskever, et al. 2018. Improving language understanding by generative pre-training. (2018)), DALL-E (Aditya Ramesh, Mikhail Pavlov, Gabriel Goh, Scott Gray, Chelsea Voss, Alec Radford, Mark Chen, and Ilya Sutskever. 2021. Zero-shot text-to-image generation. In International Conference on Machine Learning. PMLR, 8821-8831), and T5 (Colin Raffel, Noam Shazeer, Adam Roberts, Katherine Lee, Sharan Narang, Michael Matena, Yanqi Zhou, Wei Li, and Peter J Liu. 2020. Exploring the limits of transfer learning with a unified text-to-text transformer. The Journal of Machine Learning Research 21, 1 (2020), 5485-5551), has inspired the development of large molecular models like SMILES transformer (Shion Honda, Shoi Shi, and Hiroki R Ueda. 2019. Smiles transformer: Pre-trained molecular fingerprint for low data drug discovery. arXiv preprint arXiv:1911.04738 (2019)), MG-BERT (Xiao-Chen Zhang, Cheng-Kun Wu, Zhi-Jiang Yang, Zhen-Xing Wu, Jia-Cai Yi, Chang-Yu Hsieh, Ting-Jun Hou, and Dong-Sheng Cao. 2021. MG-BERT: leveraging unsupervised atomic representation learning for molecular property prediction. Briefings in bioinformatics 22, 6 (2021), bbabl52), and ChemBERT (Seyone Chithrananda, Gabriel Grand, and Bharath Ramsundar. 2020. Chemberta: Large-scale self-supervised pretraining for molecular property prediction. arXiv preprint arXiv:2010.09885 (2020)) to solve complex biomedical (John Jumper, Richard Evans, Alexander Pritzel, Tim Green, Michael Figurnov, Olaf Ronneberger, Kathryn Tunyasuvunakool, Russ Bates, Augustin Židek, Anna Potapenko, et al. 2021. Highly accurate protein structure prediction with AlphaFold. Nature 596, 7873 (2021), 583-589) and biochemical (Wenhao Gao and Connor W Coley. 2020. The synthesizability of molecules proposed by generative models. Journal of chemical information and modeling 60, 12 (2020), 5714-5723) problems. Using unsupervised or self-supervised methods, these models learn molecular fingerprints by pretraining sequence-to-sequence language models on SMILES data (John J Irwin and Brian K Shoichet. 2005. ZINCa free database of commercially available compounds for virtual screening. Journal of chemical information and modeling 45, 1 (2005), 177-182, and Sunghwan Kim, Paul A Thiessen, Evan E Bolton, Jie Chen, Gang Fu, Asta Gindulyte, Lianyi Han, Jane He, Siqian He, Benjamin A Shoemaker, et al. 2016. PubChem substance and compound databases. Nucleic acids research 44, D1 (2016), D1202-D1213). SMILES (‘simplified molecular-input line-entry system, (David Weininger. 1988. SMILES, a chemical language and information system. 1. Introduction to methodology and encoding rules. Journal of Chemical Information and Computer Sciences 28, 1 (1988), 31-36. https://doi.org/10.1021/ci00057a005)) is a text-based standard representation for molecules that is commonly used in computational chemistry. While the effectiveness of molecular foundation models such as ChemBERT and SMILES transformer has been extensively investigated in the domains of drug discovery and quantitative structure-property relationship (QSPR) prediction, their potential application for smell perception, also known as quantitative structure-odor relationship (QSOR) prediction, remains unexplored in prior research.

Recently, the QSOR problem has been approached using fully supervised training (see e.g., Justin Gilmer, Samuel S Schoenholz, Patrick F Riley, Oriol Vinyals, and George E Dahl. 2017. Neural message passing for quantum chemistry. In International conference on machine learning. PMLR, 1263-1272, Kathrin Kaeppler and Friedrich Mueller. 2013. Odor classification: a review of factors influencing perception-based odor arrangements. Chemical senses 38, 3 (2013), 189-209, Andreas Keller and Leslie B Vosshall. 2016. Olfactory perception of chemically diverse molecules. BMC neuroscience 17, 1 (2016), 1-17, Benjamin Sanchez-Lengeling, Jennifer N Wei, Brian K Lee, Richard C Gerkin, Alan Aspuru-Guzik, and Alexander B Wiltschko. 2019. Machine learning for scent: Learning generalizable perceptual representations of small molecules. arXiv preprint arXiv:1910.10685 (2019), Roberto Todeschini and Viviana Consonni. 2009. Molecular descriptors for chemoinformatics. 1. Alphabetical listing. Wiley-VCH, and Ngoc Tran, Daniel Kepple, Sergey Shuvaev, and Alexei Koulakov. 2019. DeepNose: Using artificial neural networks to represent the space of odorants. In International Conference on Machine Learning. PMLR, 6305-6314.). Keller et al. (Andreas Keller and Leslie B Vosshall. 2016. Olfactory perception of chemically diverse molecules. BMC neuroscience 17, 1 (2016), 1-17) conducted an empirical study to investigate the physical properties of molecules that evoke specific smells such as “floral” or “pungent”.

Roberto et al. (Roberto Todeschini and Viviana Consonni. 2009. Molecular descriptors for chemoinformatics. 1. Alphabetical listing. Wiley-VCH) proposed a distinct set of physico-chemical features that contribute to different smell perceptions, highlighting the role of sulfur atoms in evoking pungent smells. The most recent work, by Benjamin and Brian et al. (Brian K Lee, Emily J Mayhew, Benjamin Sanchez-Lengeling, Jennifer N Wei, Wesley W Qian, Kelsie Little, Matthew Andres, Britney B Nguyen, Theresa Moloy, Jane K Parker, et al. 2022. A Principal Odor Map Unifies Diverse Tasks in Human Olfactory Perception. bioRxiv (2022), 2022-09, and Benjamin Sanchez-Lengeling, Jennifer N Wei, Brian K Lee, Richard C Gerkin, Alan Aspuru-Guzik, and Alexander B Wiltschko. 2019. Machine learning for scent: Learning generalizable perceptual representations of small molecules. arXiv preprint arXiv:1910.10685 (2019)) employed a graph neural network (GNN) trained on molecular graphs to model odor perception. While olfactory perception has been studied long before, most classical approaches (such as Rafi Haddad, Rehan Khan, Yuji K Takahashi, Kensaku Mori, David Harel, and Noam Sobel. 2008. A metric for odorant comparison. Nature methods 5, 5 (2008), 425-429, Aharon Ravia, Kobi Snitz, Danielle Honigstein, Maya Finkel, Rotem Zirler, Ofer Perl, Lavi Secundo, Christophe Laudamiel, David Harel, and Noam Sobel. 2020. A measure of smell enables the creation of olfactory metamers. Nature 588, 7836 (2020), 118-123, and Karen J Rossiter. 1996. Structureodor relationships. Chemical reviews 96, 8 (1996), 3201-3240) rely on empirical studies to establish the relationship between molecular structure and odor descriptors. Despite these efforts, the precise connection between molecular structure and olfactory perception remains unclear. Furthermore, all of these fully-supervised methods are data-intensive, posing challenges in acquiring sufficient training data. The largest publicly available olfactory perceptual dataset, Goodscent (Fragrance Flavor. [n. d.]. Food, and Cosmetics Ingredients information. The Good Scents Company), contains only 4626 labeled samples. Even a model trained on the entire dataset from scratch through a fully-supervised method still performs poorly, particularly on sparsely represented classes. Similar to other olfactory datasets, the label distribution of the Goodscent dataset is highly skewed, as illustrated in FIG. 4. Certain odor descriptors, such as “fruity” and “sweet”, are used more frequently than others, such as “tea” and “strawberry”.

FIG. 4 shows a diagram of a distribution of perceptual descriptors of odorants on the entire dataset. Few descriptors, such as “fruity”, and “sweet”, are more often used to describe odor perception than other descriptors.

In the following, the challenges of data scarcity and label skewness are addressed by leveraging multimodal transfer learning. Specifically, it is investigated how large molecular foundation models trained on extensive unlabeled molecular data such as PubChem and ZINC (John J Irwin and Brian K Shoichet. 2005. ZINCa free database of commercially available compounds for virtual screening. Journal of chemical information and modeling 45, 1 (2005), 177-182, and Sunghwan Kim, Paul A Thiessen, Evan E Bolton, Jie Chen, Gang Fu, Asta Gindulyte, Lianyi Han, Jane He, Siqian He, Benjamin A Shoemaker, et al. 2016. PubChem substance and compound databases. Nucleic acids research 44, D1 (2016), D1202-D1213) can be potentially used to model olfactory perception effectively. Furthermore, it is explored how combining different modalities-including (a) molecular graphs that capture the symmetry and orientation of atomic systems and (b) SMILES, a sequential text encoding of chemical formulae that enables the utilization of language models—contributes effectively to developing a data-efficient perceptual model. Unlike conventional multimodal learning approaches, a naive fusion of different modalities may turn ineffective when dealing with high-dimensional and highly class-imbalanced data. To address this challenge, a label-balancer is introduced, a technique for high-dimensional multi-label and multi-modal training frameworks. The proposed label-balancer technique distributes learning objectives across different modalities, allowing different models to optimize collaboratively for distinct subsets of labels. The proposed approach leads to improved generalization and overall performance compared to single-modality training or traditional multi-modality fusion approaches (Tadas Baltrušaitis, Chaitanya Ahuja, and Louis-Philippe Morency. 2018. Multimodal machine learning: A survey and taxonomy. IEEE transactions on pattern analysis and machine intelligence 41, 2 (2018), 423-443.). The performance gain from label-balancer is especially pronounced on rare-class samples.

Various examples of the present disclosure introduce a data-efficient perceptual model through the utilization of multimodal transfer learning. It is shown that transfer features derived from pre-trained molecular foundation models are highly effective in perception modeling, even without prior training on perceptual labels. Remarkably, the proposed approach or method achieves comparable performance using only 20% of the available labeled data. This results in a substantial reduction of data requirements by 75% compared to non-transfer learning approaches. Additionally, it is explored how different modality molecular representations contribute to olfactory perception modeling.

To address the problem of skewed label distribution, the label-balancer technique that improves model generalization and performance without any additional computational cost or training data requirements is introduced.

Finally, a comprehensive evaluation of the proposed method is performed, demonstrating its performance in comparison to prior approaches across diverse experimental scenarios. Benefiting from pre-training on large unlabeled data and combining two modalities, an example implementation of the proposed framework trained via MolCLR (Yuyang Wang, Jianren Wang, Zhonglin Cao, and Amir Barati Farimani. 2022. Molecular contrastive learning of representations via graph neural networks. Nature Machine Intelligence 4, 3 (2022), 279-287) and SMILES-transformer (Shion Honda, Shoi Shi, and Hiroki R Ueda. 2019. Smiles transformer: Pre-trained molecular fingerprint for low data drug discovery. arXiv preprint arXiv:1911.04738 (2019)) demonstrates better performance on olfactory perception modeling tasks in comparison to prior supervised learning methods.

Other work can be broadly classified into three categories: olfactory perceptual model, transfer learning in olfaction, multimodal perception learning. Representative techniques in each category are introduced in the following, and the unique aspects of the proposed approach are compared to existing methods.

In the following, olfactory perceptual models are discussed. Several studies utilize machine learning empowered tools to model olfactory perception (E Dario Gutidrrez, Amit Dhurandhar, Andreas Keller, Pablo Meyer, and Guillermo A Cecchi. 2018. Predicting natural language descriptions of monomolecular odorants. Nature communications 9, 1 (2018), 4979, Yuji Nozaki and Takamichi Nakamoto. 2018. Predictive modeling for odor character of a chemical using machine learning combined with natural language processing. PloS one 13, 6 (2018), e0198475, and Ngoc Tran, Daniel Kepple, Sergey Shuvaev, and Alexei Koulakov. 2019. DeepNose: Using artificial neural networks to represent the space of odorants. In International Conference on Machine Learning. PMLR, 6305-6314.). Olfactory perception is based on perceived chemical stimuli, which are associated with complex physicochemical parameters of chemicals. Earlier studies investigated the relationships between the odor characteristics of chemicals and their physicochemical parameters using linear modeling approaches, including principal component analysis (PCA) and nonnegative matrix factorization (NMF) (Jason B Castro, Arvind Ramanathan, and Chakra S Chennubhotla. 2013. Categorical dimensions of human odor descriptor space revealed by non-negative matrix factorization. PloS one 8, 9 (2013), e73289, and Rehan M Khan, Chung-Hay Luk, Adeen Flinker, Amit Aggarwal, Hadas Lapid, Rafi Haddad, and Noam Sobel. 2007. Predicting odor pleasantness from odorant structure: pleasantness as a reflection of the physical world. Journal of Neuroscience 27, 37 (2007), 10015-10023.). However, considering the fundamentally nonlinear nature of the biological olfactory system, the suitability of these linear modeling techniques for accurately modeling olfactory perception is to be questioned. Nozaki et al. (Yuji Nozaki and Takamichi Nakamoto. 2018. Predictive modeling for odor character of a chemical using machine learning combined with natural language processing. PloS one 13, 6 (2018), e0198475) utilize nonlinear dimensionality reduction on mass spectra data as inputs and use the language modeling method word2vec to predict odor characters of chemicals.

In addition to the information from mass spectrometry, subsequent studies incorporated additional chemical structure information as explanatory variable to improve the accuracy. Traditional hand-crafted molecular representations such as Dragon (Roberto Todeschini and Viviana Consonni. 2009. Molecular descriptors for chemoinformatics. 1. Alphabetical listing. Wiley-VCH) and Mordred (Hirotomo Moriwaki, Yu-Shi Tian, Norihito Kawashita, and Tatsuya Takagi. 2018. Mordred: a molecular descriptor calculator. Journal of cheminformatics 10, 1 (2018), 1-14) are characterized by fixed-length vectors representing different physical and chemical properties of molecules. Gutierrez et al. (E Dario Gutidrrez, Amit Dhurandhar, Andreas Keller, Pablo Meyer, and Guillermo A Cecchi. 2018. Predicting natural language descriptions of monomolecular odorants. Nature communications 9, 1 (2018), 4979) predicted up to 70 olfactory perceptual descriptors using chemoinformatic features generated by Dragon. Without using cheminformatics features, Tran et al. (Ngoc Tran, Daniel Kepple, Sergey Shuvaev, and Alexei Koulakov. 2019. DeepNose: Using artificial neural networks to represent the space of odorants. In International Conference on Machine Learning. PMLR, 6305-6314) hypothesized that chemicals play the role of ligands with 3D spatial structures to olfactory receptors and, therefore, can be learned using convolutional neural networks. They trained a convolutional auto-encoder, called DeepNose, to learn the mapping between a low-dimensional 3D spatial representation of molecules and human perceptual responses. Most recently, Sanchez-Lengeling et al. (Benjamin Sanchez-Lengeling, Jennifer N Wei, Brian K Lee, Richard C Gerkin, Alan Aspuru-Guzik, and Alexander B Wiltschko. 2019. Machine learning for scent: Learning generalizable perceptual representations of small molecules. arXiv preprint arXiv:1910.10685 (2019)) trained a graph neural network to predict the relationship between a molecule's structure and its smell. The graph embeddings capture meaningful structures on both a local and global scale, which is useful in downstream QSOR tasks. Lee et al. (Brian K Lee, Emily J Mayhew, Benjamin Sanchez-Lengeling, Jennifer N Wei, Wesley W Qian, Kelsie Little, Matthew Andres, Britney B Nguyen, Theresa Moloy, Jane K Parker, et al. 2022. A Principal Odor Map Unifies Diverse Tasks in Human Olfactory Perception. bioRxiv (2022), 2022-09) extends Sanchez-Lengeling et al.'s work by employing a GNN (Graph Neural Network) to generate a Principal Odor Map (POM) that preserves and represents known perceptual relationships and enables odor quality prediction for novel odorants. However, none of these prior works explore multimodal transfer learning to address the problem of data efficiency and performance generalization, which are inherent in the olfactory domain.

In the following, transfer learning in olfaction is discussed. Unlike the olfactory perception task (QSOR prediction), several fields in the biomedical and biochemical domains explore the utility of large molecular foundation models for a wide range of tasks. Using transfer learning, chemical language models have demonstrated their capability to learn specific chemical features from a much smaller training set. Several works develop large language models similar to BERT through self-supervised training on SMILES sequences (SMILES-BERT (Sheng Wang, Yuzhi Guo, Yuhong Wang, Hongmao Sun, and Junzhou Huang. 2019.

SMILES-BERT: large scale unsupervised pre-training for molecular property prediction. In Proceedings of the 10th ACM international conference on bioinformatics, computational biology and health informatics. 429-436), MOLBERT (Benedek Fabian, Thomas Edlich, Héléna Gaspar, Marwin Segler, Joshua Meyers, Marco Fiscato, and Mohamed Ahmed. 2020. Molecular representation learning with language models and domain-relevant auxiliary tasks. arXiv preprint arXiv:2011.13230 (2020)), Bio-Bert (Jinhyuk Lee, Wonjin Yoon, Sungdong Kim, Donghyeon Kim, Sunkyu Kim, Chan Ho So, and Jaewoo Kang. 2020. BioBERT: a pre-trained biomedical language representation model for biomedical text mining. Bioinformatics 36, 4 (2020), 1234-1240), Chem-BERTa (Seyone Chithrananda, Gabriel Grand, and Bharath Ramsundar. 2020. Chemberta: Large-scale self-supervised pretraining for molecular property prediction. arXiv preprint arXiv:2010.09885 (2020)), and ChemBERTa-2 (Walid Ahmad, Elana Simon, Seyone Chithrananda, Gabriel Grand, and Bharath Ramsundar. 2022. Chemberta-2: Towards chemical foundation models. arXiv preprint arXiv:2209.01712 (2022).)). After pretraining, these models are fine-tuned for their respective downstream tasks. On the other hand, the seq2seq model is also proposed to provide effective vector representations by leveraging a large pool of unlabeled data. SMILES2Vec is an interpretable general-purpose deep neural network for predicting various chemical properties, such as toxicity, activity, solubility, and solvation energy (Garrett B Goh, Nathan O Hodas, Charles Siegel, and Abhinav Vishnu. 2017. Smiles2vec: An interpretable general-purpose deep neural network for predicting chemical properties. arXiv preprint arXiv:1712.02034 (2017)). Among the large pretrained seq2seq models, Seq3seq (Xiaoyu Zhang, Sheng Wang, Feiyun Zhu, Zheng Xu, Yuhong Wang, and Junzhou Huang. 2018. Seq3seq fingerprint: towards end-to-end semi-supervised deep drug discovery. In Proceedings of the 2018 ACM international conference on bioinformatics, computational biology, and health informatics. 404-413) is the first semi-supervised learning model for molecular property prediction. It utilizes an Encoder-Decoder structure which can provide a strong molecular representation using a huge training data pool containing a mixture of both unlabeled and labeled molecules.

SMILES Transformer using a Transformer-based seq2seq architecture is another noteworthy approach introduced by Honda et al. (Shion Honda, Shoi Shi, and Hiroki R Ueda. 2019. Smiles transformer: Pre-trained molecular fingerprint for low data drug discovery. arXiv preprint arXiv:1911.04738 (2019)). It works well for the defined downstream predictive task, especially demonstrating improved performance in small data settings. The question of whether large language models, such as GPT-3, trained on non-chemical corpora, can acquire meaningful knowledge in the field of chemistry has also been investigated in a recent study (Andrew D White, Glen M Hocky, Heta A Gandhi, Mehrad Ansari, Sam Cox, Geemi P Wellawatte, Subarna Sasmal, Ziyue Yang, Kangxin Liu, Yuvraj Singh, et al. 2022. Do large language models know chemistry?(2022)).

Besides chemical language models, molecular graphs have been widely used for pretraining strategies. Hu et al. (Weihua Hu, Bowen Liu, Joseph Gomes, Marinka Zitnik, Percy Liang, Vijay Pande, and Jure Leskovec. 2019. Strategies for pre-training graph neural networks. arXiv preprint arXiv:1905.12265 (2019)) propose to train a GNN model with context prediction or attribute masking self-supervised tasks. In the context prediction approach, a binary classifier is employed to determine whether a specific atom environment corresponds to a particular context graph. On the other hand, attribute masking involves masking random nodes, and the objective is to predict their attributes, such as atom type. Wang et al. (Yuyang Wang, Jianren Wang, Zhonglin Cao, and Amir Barati Farimani. 2022. Molecular contrastive learning of representations via graph neural networks. Nature Machine Intelligence 4, 3 (2022), 279-287) extend the pre-training with an alternative strategy based on contrastive learning. Benefiting from the different augmentation strategies they used, the fine-tuned MOLCLR (Molecular Contrastive Learning of Representations via Graph Neural Networks) model achieved state-of-the-art performance on various chemical tasks, including molecular property prediction. Despite several efforts in diverse domains, none of the prior works explore the utility of pre-trained transfer features for QSOR tasks.

In the following, multi-modal perception learning is discussed. As human beings, our perception of the environment is shaped by the information we gather through multimodal multisensory clues. A learning agent that aims to replicate human-like capabilities should also possess the ability to comprehend and generate information across different modalities. In order to learn representations of multimodal data, Silva et al. (Rui Silva, Miguel Vasco, Francisco S Melo, Ana Paiva, and Manuela Veloso. 2019. Playing games in the dark: An approach for cross-modality transfer in reinforcement learning. arXiv preprint arXiv:1911.12851 (2019)) propose to learn common encoded features using multimodal VAE (MVAE). Another approach, the Multimodal Factorization Model (MFM) (Yao-Hung Hubert Tsai, Paul Pu Liang, Amir Zadeh, Louis-Philippe Morency, and Ruslan Salakhutdinov. 2018. Learning factorized multimodal representations. arXiv preprint arXiv:1806.06176 (2018)), proposes the factorization of the multimodal representation into separate, independent representations. Vasco et al. (Miguel Vasco, Hang Yin, Francisco S Melo, and Ana Paiva. 2021. How to sense the world: Leveraging hierarchy in multimodal perception for robust reinforcement learning agents. arXiv preprint arXiv:2110.03608 (2021)) proposed a hierarchical design, called MUSE, to learn a hierarchical multimodal representation, beginning with low-level modality-specific representations from raw observation data and ending with a high-level multimodal representation encoding joint-modality information.

In the perception domain, vision and audio constitute a major part of multi-modal perception learning (Tadas Baltrusaitis, Chaitanya Ahuja, and Louis-Philippe Morency. 2018. Multimodal machine learning: A survey and taxonomy. IEEE transactions on pattern analysis and machine intelligence 41, 2 (2018), 423-443.). Chen et al. (Sihan Chen, Xingjian He, Longteng Guo, Xinxin Zhu, Weining Wang, Jinhui Tang, and Jing Liu. 2023. Valor: Vision-audio-language omni-perception pretraining model and dataset. arXiv preprint arXiv:2304.08345 (2023)) propose a Vision-Audio-Language Omni-perception pretraining model (VALOR) for multi-modal understanding and generation. Experiments show that VALOR can learn strong multimodal correlations and be generalized to various downstream tasks. In addition to visual and auditory, individuals that interact with the physical world, such as robots, will benefit from a fine-grained tactile perception of objects and surfaces. Gao et al. (Yang Gao, Lisa Anne Hendricks, Katherine J Kuchenbecker, and Trevor Darrell. 2016. Deep learning for tactile understanding from visual and haptic data. In 2016 IEEE International Conference on Robotics and Automation (ICRA). IEEE, 536-543) propose a method of classifying surfaces with haptic adjectives from both visual and physical interaction data such as friction and vibration signals. Kumari et al. (Kumari Priyadarshini, Siddhartha Chaudhuri, and Subhasis Chaudhuri. 2019. PerceptNet: Learning Perceptual Similarity of Haptic Textures in Presence of Unorderable Triplets. In IEEE World Haptics Conference (WHC)) proposed a deep neural network-based model of tactile perception that projects multiple sets of signals into the perceptual embedding space such as haptically similar material surfaces are placed closer to each other. Richard et al.'s (Richard Zhang, Phillip Isola, Alexei A Efros, Eli Shechtman, and Oliver Wang. 2018. The unreasonable effectiveness of deep features as a perceptual metric. In Proceedings of the IEEE conference on computer vision and pattern recognition. 586-595.) work is similar to the present disclosure in showing the effectiveness of deep pre-trained features for visual perception modeling tasks. However, to the best of our knowledge, the present disclosure is the first to investigate the potential of incorporating multiple modalities and transfer features in the olfactory perception domain. This unique approach has the potential to provide valuable insights into the complex interplay between chemical features and human olfactory perception, opening up new avenues for understanding and improving odor perception models.

In the following, the proposed method (according to various examples) is described. In this section, the process of extracting deep features from the molecular foundation model and calibrating them for the smell perception task is described. Next, the method of combining various modalities and training the multimodal framework in a highly dimensional and highly skewed label space, utilizing the label balancer technique is discussed. The description begins by describing some standard molecular representations that are commonly used in machine learning applications.

The most commonly used molecular features for perceptual tasks are Dragon (Roberto Todeschini and Viviana Consonni. 2009. Molecular descriptors for chemoinformatics. 1. Alphabetical listing. Wiley-VCH) and Mordred (Hirotomo Moriwaki, Yu-Shi Tian, Norihito Kawashita, and Tatsuya Takagi. 2018. Mordred: a molecular descriptor calculator. Journal of cheminformatics 10, 1 (2018), 1-14). They are a collection of several types of molecular information in tabular form, describing physical or chemical properties of a molecule, such as the atom density, the number of carbon or sulfur atoms, and the acid/base count. Mordred features, being open-sourced, are more widely used in prior studies. Other representations include molecular graphs, which capture atomic system symmetry and orientation, and SMILES, a sequential text encoding of chemical formulae that enables the use of language models.

In the following, perceptually calibrated transfer features are discussed, beginning with SMILES-based perceptual features: In order to generate pretrained deep features, the SMILES-transformer (Paul Morris, Rachel St. Clair, William Edward Hahn, and Elan Barenholtz. 2020. Predicting binding from screening assayS with transformer network embeddings. Journal of Chemical Information and Modeling 60, 9 (2020), 4191-4199) is leveraged, which has been trained on 83M molecules from the PubChem (Sunghwan Kim, Paul A Thiessen, Evan E Bolton, Jie Chen, Gang Fu, Asta Gindulyte, Lianyi Han, Jane He, Siqian He, Benjamin A Shoemaker, et al. 2016. PubChem substance and compound databases. Nucleic acids research 44, D1 (2016), D1202-D1213) repository (i.e., one of the largest repositories of molecules, consisting of comprehensive information on molecular structure and properties). Prior approaches do not make use of this vast unlabeled dataset and large language models for the QSOR task that involves learning a mapping function between the molecule's structure and its smell perception. The SMILES transformer (shown in FIG. 5) has a standard transformer architecture with six encoder and decoder layers and eight attention heads and is trained on a self-supervised task involving SMILES-IUPAC translation.

FIG. 5 shows a schematic diagram of a SMILES transformer trained through the self-supervised SMILES-IUPAC translation task. The learned embedded features are obtained from the encoder layer shown with the Smile-Transformer fingerprint label. The SMILES transformer comprises an input layer for inputting the SMILES string, two transformer encoder layers, the aforementioned Smile-Transformer fingerprint layer, two transformer decoder layers, and one output layer for outputting the predicted IUPAC names.

IUPAC is an alternative text-based representation of molecular structure, describing similar aspects of molecular structure as SMILES but using a different nomenclature. During the training process, batches of 96 molecular string pairs are used, and the Adam optimization algorithm is applied with an initial learning rate of 1e-3. The learning rate follows a cosine function within each epoch, decreasing by two orders of magnitude after completing half a period. The training is performed over 83M molecules, lasting for three epochs.

The intermediate features obtained from the pre-trained network effectively capture the information shared across both SMILES and IUPAC representations. To further refine these transfer features for olfactory perception, perceptual calibration may be performed by finetuning the MLP head using supervision derived from olfactory perceptual descriptors. This learning technique leads to learning a more refined and optimized perceptual space, with higher weights for perceptually relevant features and lower weights for less relevant ones. It may be considered important to note that the SMILES-transformer (Paul Morris, Rachel St. Clair, William Edward Hahn, and Elan Barenholtz. 2020. Predicting binding from screening assayS with transformer network embeddings. Journal of Chemical Information and Modeling 60, 9 (2020), 4191-4199) is not trained using perceptual labels. The proposed approach only fine-tunes the MLP head to facilitate the downstream task of predicting QSORs. Experimental results demonstrate that transfer features, even without explicit optimization for the perceptual task, are remarkably effective for the QSOR task. The transfer features require significantly less labeled data than the existing supervised approaches to achieve comparable performance.

In the following, graph-based perceptual features are discussed. Although the SMILES-based representation effectively encodes sequential and structural properties, it fails to capture the crucial molecular topology. Given the vastness of the chemical space, it becomes challenging for any single molecular representation to generalize across a wide range of molecules. To have better coverage of the representational space, an alternative modality representation, a molecular graph, is explored, which adequately captures the topology and structural orientation of molecules. Similar to SMILES, a pre-trained model, MolCLR (Yuyang Wang, Jianren Wang, Zhonglin Cao, and Amir Barati Farimani. 2022. Molecular contrastive learning of representations via graph neural networks. Nature Machine Intelligence 4, 3 (2022), 279-287), trained on 10M PubChem molecules through self-supervised contrastive loss, is employed.

FIG. 6 shows a schematic diagram of the MolCLR framework optimized for olfactory perception task. the highlight mask shows the removed sub-graph resulting from the graph augmentation technique. the final layer embedding features, denoted as zi, are fine-tuned for the downstream QSOR task.

MolCLR defines the self-supervised task using three different graph augmentation techniques: atom masking, bond deletion, and subgraph removal. The positive pairs constitute a molecule and its corresponding augmented molecule graph, while any two different molecules form negative pairs. Similar to the SMILES-transformer, our graph framework consists of pre-trained MolCLR and MLP head, and the MLP head is finetuned for downstream QSOR tasks using perceptual labels. MolCLR uses a 5-layer graph convolution (Thomas N Kipf and Max Welling. 2016. Semi-supervised classification with graph convolutional networks. arXiv preprint arXiv:1609.02907 (2016)) with ReLU activation as the GNN backbone, incorporating modifications from Hu et al. (Weihua Hu, Bowen Liu, Joseph Gomes, Marinka Zitnik, Percy Liang, Vijay Pande, and Jure Leskovec. 2019. Strategies for pre-training graph neural networks. arXiv preprint arXiv:1905.12265 (2019)) to support edge features. Graph-level readout is performed through average pooling, producing a 512-dimensional molecular representation. The NT-Xent loss is optimized using the Adam optimizer with weight decay 10e-5. The model is trained for a total of 50 epochs with a batch size of 512.

In the following, multimodal representation is discussed. The present disclosure explores how combining the graph with text-based molecular representations helps learn more effective perceptual features. The proposed method draws inspiration from ensemble learning approaches. Combining diverse modalities or models usually improves the performance of machine learning methods. This improvement becomes more pronounced when the features or models are dissimilar from each other, as they contribute uniquely to the learning process.

Multimodal fusion offers several advantages—a) multimodal information may offer complementary information for the defined learning task, b) multimodal learning can be viewed as an ensemble learning approach where multiple models optimize for the same downstream task, resulting in improved and robust performance, and c) certain modalities may be more expensive to obtain than others, in which case multimodal learning can still operate even in the absence of one or a few modalities. The present disclosure begins by investigating the effectiveness of classical fusion methods by optimizing uni-modal SMILE transfer features z_{S}, and graph transfer features z_{G}, individually as well as jointly using static fusion approaches such as concatenation v_{S}∥z_{G}, element-wise sum z_{S}⊕vG, and product z_{S}⊙z_{G}. The final embedding features, after combining the modalities using an element-wise sum, can be expressed as follows:

$${z}_{M} = {f}_{M} ( {f}_{S} ( {z}_{S} ) ⊕ {f}_{G} ( {z}_{G} ) )$$

Here, f_{S} and f_{G} represent the MLP heads for SMILE-transformer and MolCLR, respectively. f_{M} refers to the final linear layer that combines different modality features with optimized or optimal weights based on their perceptual relevance.

FIG. 7 shows a schematic diagram of the label balancer technique. The label balancer minimizes the impact of the skewed dataset and helps achieve better performance and generalization on all class samples. The sets y_{S} and y_{G} denote subsets of labels optimized by SMILES-transformer and MolCLR, respectively. After each training round of weight updates, both modality features are combined using a static fusion approach.

In the following, the label-balancer is discussed. During development, it was observed that the final multimodal features may be less effective due to the high correlation between modalities. This correlation may lead to a reduced amount of complementary information available to aid the models. Furthermore, training may become even more challenging with a high-dimensional skewed label distribution. To address this challenge, the label-balancer training technique was introduced, which mitigates overfitting and offers better generalization on rare-class test samples. The core idea of some examples is to distribute learning objectives across different modalities, enabling different models to optimize collaboratively for distinct subsets of labels. For example, in each training iteration, a random label division strategy may be used, where half of the labels are optimized using the SMILES transformer, and the remaining half are optimized using the MolCLR. For example, the following equation may be used as objective function:

$${L}_{c e} = - {\sum }_{i = 1}^{L} log {p}_{l}^{y} {𝕀}_{{y}^{s}} ( i ) - log {p}_{l}^{y} {𝕀}_{{y}^{G}} ( i )$$

Here y_{S} and y_{G} are complementary sets, denoting label subsets optimized by SMILES-transformer and MolCLR, respectively. The division of labels among different models enables each model to learn more effective features for the assigned labels. Moreover, by integrating diverse features learned from distinct models trained on different label sets, our method demonstrates improved generalization capability, which is particularly difficult to achieve with high-dimensional multi-label data. The proposed training framework improves performance compared to uni-modal training and classical multi-modality fusion approaches (Tadas Baltrušaitis, Chaitanya Ahuja, and Louis-Philippe Morency. 2018. Multimodal machine learning: A survey and taxonomy. IEEE transactions on pattern analysis and machine intelligence 41, 2 (2018), 423-443.). Further improvement in performance is anticipated as additional modalities are integrated into the training framework.

In the following, the proposed concept is evaluated. The framework is evaluated through several experiments addressing three questions: (Q1) How effective are pre-trained molecular foundation models for modeling olfactory perception?(Q2) Does combining different molecular representations, such as molecular graphs and text-based SMILES, result in better perceptual features?(Q3) How effective is our multimodal training technique, label-balancer, compared to classical fusion techniques for high-dimensional and highly skewed multi-label spaces? The introduction is started by introducing the dataset, implementation details, and evaluation setup before discussing each question in subsequent subsections.

Dataset. In the olfactory domain, there are very few perception datasets. The commonly used datasets include the Dravenieks database (Andrew Dravnieks et al. 1985. Atlas of odor character profiles), which comprises only 138 molecules described by a 131D dimensional perceptual label vector. Additionally, the Keller dataset (Andreas Keller and Leslie B Vosshall. 2016. Olfactory perception of chemically diverse molecules. BMC neuroscience 17, 1 (2016), 1-17) consists of 480 molecules, with non-expert provided 20D dimensional descriptors. Other notable datasets are the Goodscents dataset (Fragrance Flavor. [n. d.]. Food, and Cosmetics Ingredients information. The Good Scents Company.), containing 4626 molecules described by 668D dimensional descriptors, and the Leffingwell dataset [LEFFINGWELL ASSOCIATES. 2001. Database of Perfumery Materials & Performance. (2001). http://www.leffingwell.com/bacispmp.htm.], consisting of 3522 molecules described by 113D dimensional descriptors. The descriptor labels for both datasets, Goodscents and Leffingwell, are gathered from domain-experts and hence are less noisy. While the Dravenieks database (Andrew Dravnieks et al. 1985. Atlas of odor character profiles) is too small to be effectively used in learning-based techniques, the Keller dataset suffers from noise and sparsity issues due to the labels being collected from non-experts. To generate a large-scale and clean dataset, after filtering out noisy labels and inconsistent molecules, a collection of 5595 molecules was compiled from the Goodscents and Leffingwell datasets, described by 91D dimensional perceptual descriptors. Even after cleaning out noisy labels, the final curated dataset has a skewed label distribution, where certain descriptors such as “fruity” are frequently used, while descriptors like “dairy” and “tea” are sparsely used. Moreover, the label set is also fine-grained, consisting of broad and commonly used descriptors like fruity to more specific labels such as apple, pear, pineapple, etc.

Implementation Details. The SMILES-transformer, consisting of six encoded and decoded layers (Shion Honda, Shoi Shi, and Hiroki R Ueda. 2019. Smiles transformer: Pre-trained molecular fingerprint for low data drug discovery. arXiv preprint arXiv:1911.04738 (2019)) and MolCLR (Yuyang Wang, Jianren Wang, Zhonglin Cao, and Amir Barati Farimani. 2022. Molecular contrastive learning of representations via graph neural networks. Nature Machine Intelligence 4, 3 (2022), 279-287), consisting of a 5-layer graph convolution with ReLU activation, were used. Both modality representations were fine-tuned using an MLP head with 512 neurons. The model was trained using the cross-entropy loss, with the Adam optimizer on a batch size of 32 for 5000 epochs. The performance of the learned model was evaluated using the AUROC (Area Under the Receiver Operating Characteristic) metric, which is commonly used for multilabel classification problems. The model's performance was measured by calculating the unweighted mean AUROC, which involves averaging the AUROC scores across all 91 odor descriptors and assigning equal weights to all descriptors to ensure unbiased performance comparison.

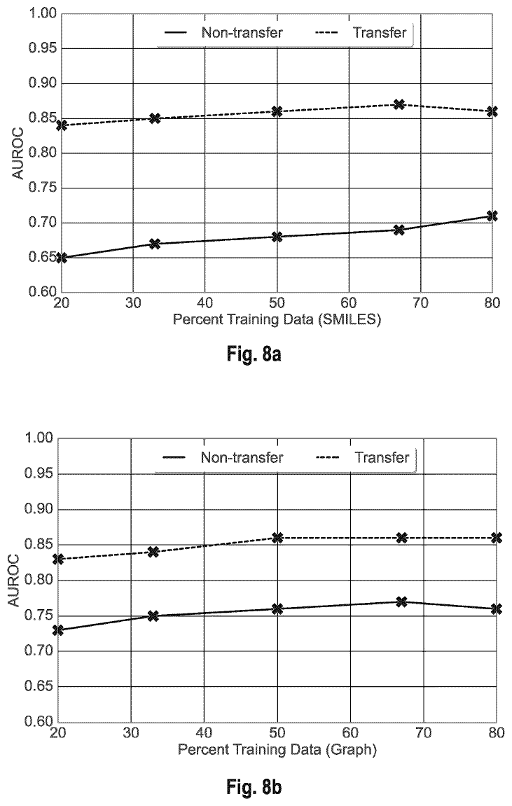

In the following, the results and their discussion are provided. Regarding, Q1 (How effective are pre-trained molecular foundation models for modeling olfactory perception?), FIGS. 8a and 8b are presented. FIGS. 8a and 8b show diagrams of the performance of perceptual features learned with and without transfer learning on (a) SMILES representation (FIG. 8a) (b) molecular graph representation (FIG. 8b) with an increasing amount of training data. For both representations, the proposed approach, leveraging pre-trained features from molecular foundation models, outperforms state-of-the-art methods by a significant margin.

FIG. 8a shows the performance comparison between perceptual features learned with and without using a pre-trained SMILES transformer model. To obtain non-transfer features, state-of-the-art by Zheng (Xiaofan Zheng, Yoichi Tomiura, and Kenshi Hayashi. 2022. Investigation of the structure-odor relationship using a Transformer model. Journal of Cheminformatics 14, 1 (2022), 88) was used. The SMILES-transformer (Xiaofan Zheng, Yoichi Tomiura, and Kenshi Hayashi. 2022. Investigation of the structure-odor relationship using a Transformer model. Journal of Cheminformatics 14, 1 (2022), 88) was trained from scratch using the perceptual training data. To evaluate whether the model's performance is limited by the amount of data, training was conducted on progressively larger volumes of data from 20% to 80% of the whole dataset. The results demonstrate that the performance of the non-transfer model improves as more data is used, but it remains suboptimal even after utilizing 80% of the available training data. However, by leveraging the pre-trained model (Shion Honda, Shoi Shi, and Hiroki R Ueda. 2019. Smiles transformer: Pre-trained molecular fingerprint for low data drug discovery. arXiv preprint arXiv:1911.04738 (2019)), the proposed method significantly outperforms the state-of-the-art. Even with just 20% of the available labeled data, considerably higher AUROC are obtained compared to the performance achieved without transfer learning.

It is noted that the pre-trained SMILES-transformer (Shion Honda, Shoi Shi, and Hiroki R Ueda. 2019. Smiles transformer: Pre-trained molecular fingerprint for low data drug discovery. arXiv preprint arXiv:1911.04738 (2019)) is trained using a self-supervised task of SMILES-IUPAC translation, which does not have an obvious connection with smell perception. Remarkably, the transfer features learned from this self-supervised objective are perceptually effective and yield substantial performance gains with minimal perceptual supervision on only 20% of the data. Moreover, the computational overhead for learning weights for just one MLP head is significantly reduced compared to training the entire model from scratch. The rate of performance improvement with increasing training data is less pronounced in the case of transfer learning. This can be attributed to the diminished potential for improvement over the already rich and effective pre-trained features generated from millions of unlabeled samples.

FIG. 8b depicts a similar comparison for the molecular graph. Non-transfer graph features were learned using another state-of-the-art approach in the molecular graph domain by Sanchez-Lengeling et al. (Benjamin Sanchez-Lengeling, Jennifer N Wei, Brian K Lee, Richard C Gerkin, Alán Aspuru-Guzik, and Alexander B Wiltschko. 2019. Machine learning for scent: Learning generalizable perceptual representations of small molecules. arXiv preprint arXiv:1910.10685 (2019)). They utilize a graph neural network to model olfactory perception and evaluate their approach using a curated Goodscents dataset. As their data is not publicly available, their model was trained on the dataset used herein to demonstrate the performance of the learned non-transfer features. As shown in FIG. 8b, the proposed method significantly outperforms the state-of-the-art. However, it is worth noting that the performance of non-transfer features derived from the molecular graph is considerably superior to that of the SMILES representation. This observation aligns with what can intuitively be expected, as the molecular graph is more effective in capturing key elements for modeling olfactory perception (such as the presence or absence of atoms, types of atomic bonds, orientation, and topology) than the text-based simpler representation provided by SMILES.

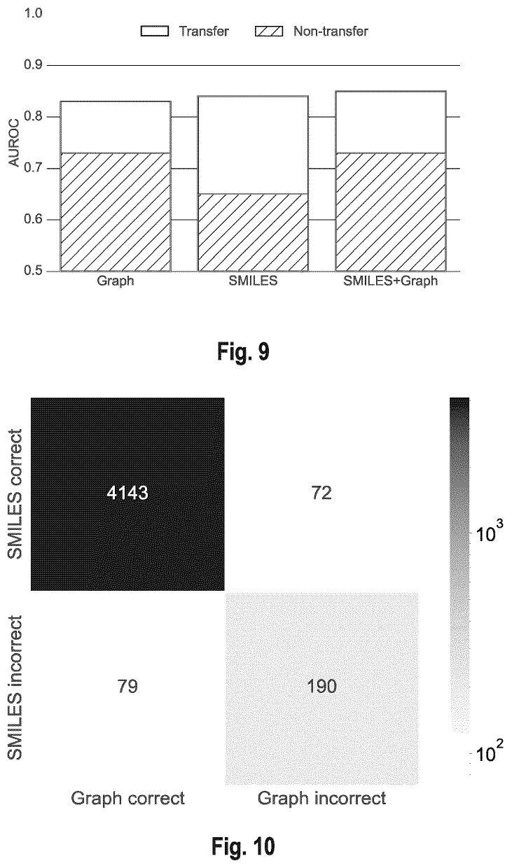

Next, the benefits of transfer learning for both unimodal and multi-modal features was examined. Similar to the previous evaluations, state-of-the-art methods (Benjamin Sanchez-Lengeling, Jennifer N Wei, Brian K Lee, Richard C Gerkin, Alan Aspuru-Guzik, and Alexander B Wiltschko. 2019. Machine learning for scent: Learning generalizable perceptual representations of small molecules. arXiv preprint arXiv:1910.10685 (2019)). Xiaofan Zheng, Yoichi Tomiura, and Kenshi Hayashi. 2022. Investigation of the structure-odor relationship using a Transformer model. Journal of Cheminformatics 14, 1 (2022), 88) were utilized for the SMILES and graph representations, when learning non-transfer features. To derive multi-modal features, the graph and SMILES representations were combined using a traditional fusion method that involves element-wise summation. As shown in FIG. 9, it is observed that the advantages of transfer learning extend beyond single-modality features. Even in the case of multi-modal features, significant performance gains are achieved through the use of transfer learning. Takeaway 1: The transfer features acquired from molecular foundation models demonstrate remarkable perceptual effectiveness and robustness across diverse feature types and datasets. FIG. 9 shows a diagram of an ablation study to show the benefit of transfer learning on uni-modal and multi-modal representations. Transfer learning helps in both uni -and multi-modal features.

Regarding Q2 (Does combining different molecular representations, such as molecular graphs and text-based SMILES, result in better perceptual features?), the following results were obtained.

In the following, a table of different uni- and multi-modal features for olfactory perception tasks is shown. For the sensitivity analysis, the performance of the fusion variations, including element-wise sum ⊕, product ⊙, and concatenation ∥ is shown.

| Features | Multimodal | Test AUROC |
|---|---|---|
| SMILES (S) | X | 0.71 |
| Graph (G) | X | 0.76 |
| (Sanchez-Lengeling) | | |
| MORDRED (M) | X | 0.80 |
| S ⊕ G | ✓ | 0.81 |
| S ⊕ M | ✓ | 0.83 |
| G ⊕ M | ✓ | 0.84 |
| S ⊕ G ⊕ M | ✓ | 0.81 |
| S ⊙ G ⊙ M | ✓ | 0.84 |
| S ∥ G ∥ M | ✓ | 0.84 |

The above table provides an overview of the performance of different uni- and multimodal features for olfactory perception tasks. For the single modality features, the proposed method was compared with the relevant state-of-the-art approaches. In all cases, the model was trained on 80% of the available data and tested on the remaining 20% of the data. The results clearly demonstrate that while there is some improvement achieved by combining modalities, the gains are relatively less significant compared to the benefits observed from transfer features. Among all the fusion variations, such as element-wise sum, product, and concatenation, the best performance was observed with the concatenation and element-wise product features.

Upon further examination of the (poor) performance of multimodal features, it was found that different modalities tend to make similar errors. Specifically, it was observed that (all) modalities make accurate predictions on samples belonging to well-represented classes, while simultaneously making errors on samples from rare classes. FIG. 10 shows the percentage of test samples where both SMILES and the graph model make correct or incorrect predictions. FIG. 10 shows a diagram of a distribution of test samples where both SMILES and graph models make (dis)similar predictions. There are only few samples where both models make different predictions. This result suggests that the individual modality is not diverse enough to contribute complementary information to the overall performance of the combined model. Takeaway 2: The integration of graph-based features and SMILES representations results in limited improvements when compared to transfer learning methods due to a lack of complementary information.

Regarding Q3 (How effective is our multimodal training technique, label-balancer, compared to classical fusion techniques for high-dimensional and highly skewed multi-label spaces?), the effectiveness of our proposed label balancer technique was evaluated and compared to classical fusion approaches for combining different modalities. To evaluate multimodality techniques, two models were trained: one utilizes the classical element-wise sum and fine-tuning with an MLP head, while the other employs the label balancer technique to train the joint model, using both SMILES and graph inputs. The performance of both models was evaluated by comparing an average test AUROC value computed across all 91 perceptual descriptors. To assess the robustness of the label balancer technique, experiments were conducted on training data of varying sizes ranging from 5% to 80%. FIG. 11 shows a diagram of a performance comparison between classical multi-modal fusion technique (MLP head) and label balancer. The result shows the average test AUROC value across all 91 perceptual descriptors. The proposed label balancer consistently outperforms the MLP head across all training dataset sizes.

In the FIG. 11, the solid curve represents the performance of the combined model trained using the MLP head, while the dashed curve represents the performance using the label balancer technique. Notably, the label balancer technique consistently outperforms the classical fusion approach across all training subsets. This further emphasizes the effectiveness and robustness of the proposed approach for multilabel multimodal training. While FIG. 11 demonstrates the effectiveness of the label balancer compared to the MLP head, the gain is normalized due to averaging AUROC across all label descriptors. Next, it is explicitly demonstrated how the label balancer technique affects the performance of well and sparsely represented classes separately.

The performance of the MLP head and the label balancer was evaluated on each class separately. For ease of visualization, all classes were grouped into clusters based on the sample density. In FIG. 12, the x-axis represents the clusters formed from all 91 descriptor classes, ranging from the most-dense class (left-most) to the most sparse class (right-most). FIG. 12 shows a diagram showing a performance gain by the label balancer over the MLP head approach on most-dense to most-sparse classes.

For each cluster, the average performance gain achieved by the label balancer over the MLP head was evaluated. As shown in FIG. 12, the label balancer technique yields higher gains on sparsely represented classes compared to densely represented classes. The increasing trend from left to right validates the intuition that training with distributed objectives across different modalities helps in the generalization of the model for rare class samples. Furthermore, the performance on the dense class is already good, leaving less room for improvement for the label balancer technique. To see the performance gain for each class, FIG. 15 may be consulted.

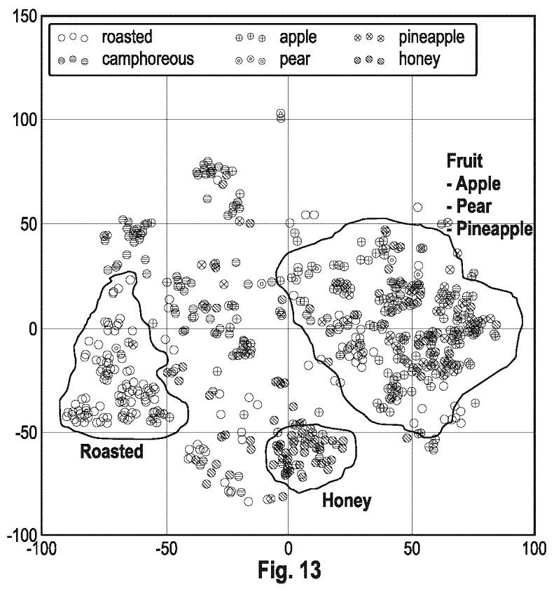

Finally, the multimodal representation learned by the proposed label balancer for combining SMILES and graph features was examined. In order to visualize the learned embedding, t-SNE algorithms were used to project the learned representation of test samples onto a 2D space. FIG. 13 shows a visualization of a molecular representation learned by the proposed model via t-SNE. Representations are shown for the test samples on randomly selected labels, which are shown by different shadings.

It is to be noted that each sample may have multiple labels, resulting in molecules belonging to different class clusters rather than a single one. As a result, diffused clusters are observed in the embedding space, in contrast to the tight clusters observed in multiclass problems where classes are mutually exclusive. Despite this, perceptually similar classes are seen appearing closer to each other than distinct ones. For instance, molecules that evoke fruit smells such as “apple”, “pear”, and “pineapple” form a cluster and are perceptual neighbors in the embedding space. Similarly, other flavors like “roasted” and “honey” are also grouped together. This observation suggests that the proposed method captures the perceptual similarity between different flavors in a meaningful manner, despite the challenges posed by the multi-label nature of the problem. Takeaway 3: The label balancer is effective in learning multimodal representation, particularly in high-dimensional and highly skewed multi-label spaces. It successfully learns perceptually meaningful representations and improves generalization, specifically for sparsely represented classes.

In the following, the conclusions are presented. The present disclosure addresses the challenges of data scarcity and label skewness in olfactory perception modeling by leveraging multimodal transfer learning. It was demonstrated that the pre-trained molecular foundation models are effective in learning olfactory perception with minimal supervision. The data scarcity problem was addressed by leveraging pre-trained features and reducing the amount of data required for training by up to 75% compared to non-transfer learning approaches. The effectiveness of different molecular representations was investigated, and the label-balancer technique was introduced to improve model generalization and performance in scenarios where the label space is high-dimensional and highly skewed. Experimental results on the largest publicly available olfactory perception dataset, Goodscents, validate that the proposed method achieves both data efficiency and robust performance compared to state-of-the-art methods.

There are several interesting research directions to explore as future work. A molecular foundation model may be built, leveraging Mordred features similar to SMILES-transfer and MolCLR. Incorporating additional modalities may further improve the performance of the label-balancer approach. The effectiveness of the label-balancer technique may be explored across a wider range of modalities and their combinations. Additionally, exploring novel approaches for multilabel and multimodal learning that exhibit robustness and generalization across different classes may be of great interest and crucial for understanding human smell perception.

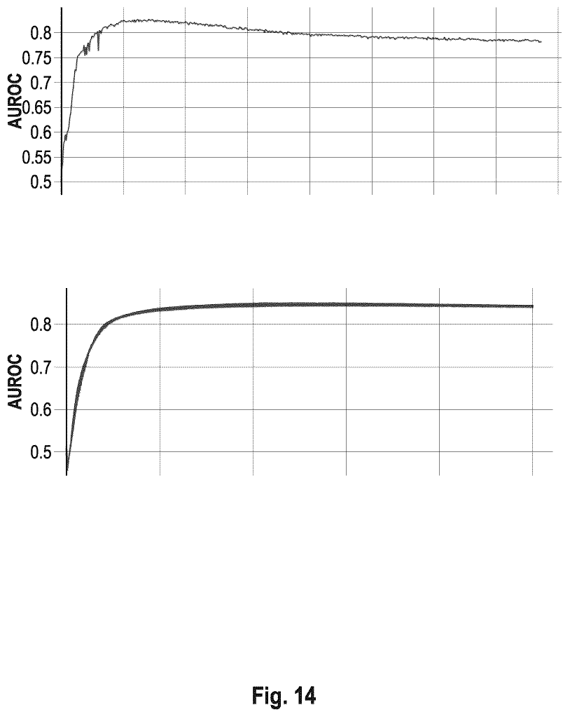

FIG. 14 show the AUROC of the MLP head fusion approach (on top) and of the label balancer approach (on the bottom). FIG. 14 (top) depicts the test AUROC of the MLP head fusion approach as the number of epochs increases, while the bottom illustrates the same with the label balancer technique. In the first case, the performance initially improves but eventually starts to deteriorate, suggesting model overfitting. Conversely, the lower curve remains consistently robust throughout the training process.

In the following, an ablation study to show the contribution of multimodal and transfer learning for modeling olfactory perception is shown. The following table shows a performance comparison with and without transfer or/and multimodal learning for modeling olfactory perception.

| Features | Transfer | Multimodal | Test AUROC |
|---|---|---|---|
| SMILES (S) | X | X | 0.71 |
| Graph (G) | X | X | 0.76 |
| MORDRED (M) | X | X | 0.80 |
| S + G | X | ✓ | 0.81 |
| S + M | X | ✓ | 0.83 |
| G + M | X | ✓ | 0.84 |
| S + G + M | X | ✓ | 0.84 |
| S + G with | ✓ | ✓ | 0.87 |
| Label Balancer | | | |

FIG. 15 shows a table of performance comparisons of label balancer and MLP head on each class.

More details and aspects of the technique for improving or optimizing learning across multimodal transfer features for modeling olfactory perception are mentioned in connection with the proposed concept or one or more examples described above or below (e.g., FIG. 1a to 3c). The technique for improving or optimizing learning across multimodal transfer features for modeling olfactory perception may comprise one or more additional optional features corresponding to one or more aspects of the proposed concept, or one or more examples described above or below.

In the following, some examples of the proposed concept are shown:
1) An apparatus for predicting an olfactory profile of a molecule, the apparatus comprising memory circuitry, machine-readable instructions, and processor circuitry to execute the machine-readable instructions to:
   obtain a first representation of the molecule and a second representation of the molecule;
   process the first representation using at least one first machine-learning model to obtain a first predicted olfactory profile of the molecule;
   process the second representation using at least one second machine-learning model to obtain a second predicted olfactory profile of the molecule; and
   process the first predicted olfactory profile and the second predicted olfactory profile, or a combined version of the first predicted olfactory profile and the second predicted olfactory profile, using a third machine-learning model, the third machine-learning model being trained to output a third predicted olfactory profile of the molecule.
2) The apparatus according to 1), wherein at least the third predicted olfactory profile represents a plurality of olfactory labels, wherein at least a component of the at least one first machine-learning model is trained to predict a first subset of the plurality of olfactory labels and at least a component of the at least one second machine-learning model is trained to predict a second subset of the plurality of olfactory labels.
3) The apparatus according to 2), wherein the third machine-learning model is trained to generate the third predicted olfactory profile based on the labels predicted by the at least one first machine-learning model and the at least one second machine-learning model.
4) The apparatus according to one of 1) to 3), wherein the processor circuitry is to execute the machine-readable instructions to combine the first predicted olfactory profile and the second predicted olfactory profile to generate an input to the third machine-learning model.
5) The apparatus according to 4), wherein the first predicted olfactory profile and the second predicted olfactory profile is combined using one of concatenation, element-wise summation, and multiplication.
6) The apparatus according to one of 1) to 5), wherein the at least one first machine-learning model and the at least one first machine-learning model each comprise a pre-trained machine-learning model to generate an embedding of the molecule and a predictor machine-learning model to predict the respective first and second predicted olfactory profile based on the respective embedding of the molecule.
7) The apparatus according to 6), wherein the pre-trained machine-learning model is trained using self-supervised training.
8) The apparatus according to one of 6) to 7), wherein at least one of the pre-trained machine-learning models is a model for generating an embedding or representation of the molecule based on a graph representation of the molecule.
9) The apparatus according to one of 6) to 8), wherein at least one of the pre-trained machine-learning models is a model for generating an embedding or representation of the molecule based on a textual representation of the molecule.
10) The apparatus according to one of 6) to 9), wherein the third machine-learning model and the predictor machine-learning models are trained together using end-to-end training.
11) The apparatus according to one of 1) to 10), wherein the first representation of the molecule is according to a first modality, and the second representation of the molecule is according to a second modality being different from the first modality.
12) The apparatus according to 11), wherein at least one of the first modality and the second modality is one of a textual representation of the molecule, a graph representation of the molecule, an image representation of the molecule and a multi-dimensional embedding of the molecule.
13) The apparatus according to one of 1) to 12), wherein the processor circuitry is to execute the machine-readable instructions to obtain at least one further representation of the molecule, process the at least one further representation using at least one further machine-learning model to obtain at least one further predicted olfactory profile of the molecule, and to process the first, second and at least one further predicted olfactory profile, or a combined version of the first, second and at least one further predicted olfactory profile using the third machine-learning model to generate the third predicted olfactory profile.
14) The apparatus according to one of 1) to 13), wherein the processor circuitry is to execute the machine-readable instructions to obtain a plurality of representations for a plurality of molecules, process the plurality of representations to obtain a plurality of third predicted olfactory profiles, and store the plurality of third olfactory profiles together with information on the respective molecule in a data structure.
15) The apparatus according to 14), wherein the processor circuitry is to execute the machine-readable instructions to select one or more molecules from the data structure based on a desired olfactory profile.
16) The apparatus according to one of 1) to 15), wherein the third olfactory profile is provided for the purpose of selecting the molecule for use in one of a perfume, perfume component for another substance, cosmetic substance, and food item.
17) The apparatus according to one of 1) to 16), wherein the processor circuitry includes at least one of a central processing unit, a graphics processing unit, an artificial intelligence accelerator, a field-programmable gate array, and an application-specific integrated circuit.
18) An apparatus for selecting a molecule, the apparatus comprising memory circuitry, machine-readable instructions, and processor circuitry to execute the machine-readable instructions to:
   select one or more molecules from a data structure based on a desired olfactory profile, with the data structure being generated by an apparatus according to 14); and
   provide information on the one or more molecules for the purpose of selecting the one or more molecules for use in one of a perfume, perfume component for another substance cosmetic substance, and food item.
19) An apparatus for training machine-learning models, the apparatus comprising memory circuitry, machine-readable instructions, and processor circuitry to execute the machine-readable instructions to:
   obtain training data, the training data comprising information on a plurality of molecules and associated olfactory profiles of the plurality of molecules;
   train at least a component of at least one first machine-learning model, at least a component of at least one second machine-learning model, and a third machine-learning model using the training data,
   wherein the at least one first machine-learning model is trained to output a first predicted olfactory profile of a molecule based on a first representation of the molecule,
   the at least one second machine-learning model is trained to output a second predicted olfactory profile of the molecule based on a second representation of the molecule, and the third machine-learning model is trained to output a third predicted olfactory profile of the molecule using the first predicted olfactory profile and the second predicted olfactory profile, or a combined version of the first predicted olfactory profile and the second predicted olfactory profile, as input.
20) The apparatus according to 19), wherein at least a component of the at least one first machine-learning model, at least a component of the at least one second machine-learning model, and the third machine-learning model are trained using supervised learning.
21) The apparatus according to one of 19) or 20), wherein at least the third predicted olfactory profile represents a plurality of olfactory labels, wherein at least a component of the at least one first machine-learning model is trained to predict a first subset of the plurality of olfactory labels and at least a component of the at least one second machine-learning model is trained to predict a second subset of the plurality of olfactory labels.
22) The apparatus according to one of 19) to 21), wherein the at least one first machine-learning model and the at least one first machine-learning model each comprise a pre-trained machine-learning model to generate an embedding of the molecule and a predictor machine-learning model to predict the respective first and second predicted olfactory profile based on the respective embedding of the molecule, wherein the third machine-learning model and the predictor machine-learning models are trained using the training data.
23) The apparatus according to 22), wherein the pre-trained machine-learning models remain unmodified when the third machine-learning model and the predictor machine-learning models are trained using the training data.
24) The apparatus according to one of 22) or 23), wherein the third machine-learning model and the predictor machine-learning models are trained together, using the training data, using end-to-end training.

The aspects and features described in relation to a particular one of the previous examples may also be combined with one or more of the further examples to replace an identical or similar feature of that further example or to additionally introduce the features into the further example.

Examples may further be or relate to a (computer) program including a program code to execute one or more of the above methods when the program is executed on a computer, processor or other programmable hardware component. Thus, steps, operations or processes of different ones of the methods described above may also be executed by programmed computers, processors or other programmable hardware components. Examples may also cover program storage devices, such as digital data storage media, which are machine-, processor- or computer-readable and encode and/or contain machine-executable, processor-executable or computer-executable programs and instructions. Program storage devices may include or be digital storage devices, magnetic storage media such as magnetic disks and magnetic tapes, hard disk drives, or optically readable digital data storage media, for example. Other examples may also include computers, processors, control units, (field) programmable logic arrayS ((F)PLAs), (field) programmable gate arrayS ((F)PGAs), graphics processor units (GPU), application-specific integrated circuits (ASICs), integrated circuits (ICs) or system-on-a-chip (SoCs) systems programmed to execute the steps of the methods described above.

It is further understood that the disclosure of several steps, processes, operations or functions disclosed in the description or claims shall not be construed to imply that these operations are necessarily dependent on the order described, unless explicitly stated in the individual case or necessary for technical reasons. Therefore, the previous description does not limit the execution of several steps or functions to a certain order. Furthermore, in further examples, a single step, function, process or operation may include and/or be broken up into several sub-steps, -functions, -processes or -operations.

If some aspects have been described in relation to a device or system, these aspects should also be understood as a description of the corresponding method. For example, a block, device or functional aspect of the device or system may correspond to a feature, such as a method step, of the corresponding method. Accordingly, aspects described in relation to a method shall also be understood as a description of a corresponding block, a corresponding element, a property or a functional feature of a corresponding device or a corresponding system.

The following claims are hereby incorporated in the detailed description, wherein each claim may stand on its own as a separate example. It should also be noted that although in the claims a dependent claim refers to a particular combination with one or more other claims, other examples may also include a combination of the dependent claim with the subject matter of any other dependent or independent claim. Such combinations are hereby explicitly proposed, unless it is stated in the individual case that a particular combination is not intended. Furthermore, features of a claim should also be included for any other independent claim, even if that claim is not directly defined as dependent on that other independent claim.

What is claimed is:

1. An apparatus for predicting an olfactory profile of a molecule, the apparatus comprising memory circuitry, machine-readable instructions, and processor circuitry to execute the machine-readable instructions to:
obtain a first representation of the molecule and a second representation of the molecule;
process the first representation using at least one first machine-learning model to obtain a first predicted olfactory profile of the molecule;
process the second representation using at least one second machine-learning model to obtain a second predicted olfactory profile of the molecule;
process the first predicted olfactory profile and the second predicted olfactory profile, or a combined version of the first predicted olfactory profile and the second predicted olfactory profile, using a third machine-learning model, the third machine-learning model being trained to output a third predicted olfactory profile of the molecule.

2. The apparatus according to claim 1, wherein at least the third predicted olfactory profile represents a plurality of olfactory labels, wherein at least a component of the at least one first machine-learning model is trained to predict a first subset of the plurality of olfactory labels and at least a component of the at least one second machine-learning model is trained to predict a second subset of the plurality of olfactory labels.

3. The apparatus according to claim 1, wherein the processor circuitry is to execute the machine-readable instructions to combine the first predicted olfactory profile and the second predicted olfactory profile to generate an input to the third machine-learning model.

4. The apparatus according to claim 3, wherein the first predicted olfactory profile and the second predicted olfactory profile is combined using one of concatenation, element-wise summation, and multiplication.

5. The apparatus according to claim 1, wherein the at least one first machine-learning model and the at least one first machine-learning model each comprise a pre-trained machine-learning model to generate an embedding of the molecule and a predictor machine-learning model to predict the respective first and second predicted olfactory profile based on the respective embedding of the molecule.

6. The apparatus according to claim 5, wherein the pre-trained machine-learning model is trained using self-supervised training.

7. The apparatus according to claim 5, wherein at least one of the pre-trained machine-learning models is a model for generating an embedding or representation of the molecule based on a graph representation of the molecule.

8. The apparatus according to claim 5, wherein at least one of the pre-trained machine-learning models is a model for generating an embedding or representation of the molecule based on a textual representation of the molecule.

9. The apparatus according to claim 5, wherein the third machine-learning model and the predictor machine-learning models are trained together using end-to-end training.

10. The apparatus according to claim 1, wherein the first representation of the molecule is according to a first modality, and the second representation of the molecule is according to a second modality being different from the first modality.

11. The apparatus according to claim 10, wherein at least one of the first modality and the second modality is one of a textual representation of the molecule, a graph representation of the molecule, an image representation of the molecule and a multi-dimensional embedding of the molecule.

12. The apparatus according to claim 1, wherein the processor circuitry is to execute the machine-readable instructions to obtain at least one further representation of the molecule, process the at least one further representation using at least one further machine-learning model to obtain at least one further predicted olfactory profile of the molecule, and to process the first, second and at least one further predicted olfactory profile, or a combined version of the first, second and at least one further predicted olfactory profile using the third machine-learning model to generate the third predicted olfactory profile.

13. The apparatus according to claim 1, wherein the processor circuitry is to execute the machine-readable instructions to obtain a plurality of representations for a plurality of molecules, process the plurality of representations to obtain a plurality of third predicted olfactory profiles, and store the plurality of third olfactory profiles together with information on the respective molecule in a data structure.

14. The apparatus according to claim 1, wherein the third olfactory profile is provided for the purpose of selecting the molecule for use in one of a perfume, perfume component for another substance, cosmetic substance, and food item.

15. The apparatus according to claim 1, wherein the processor circuitry includes at least one of a central processing unit, a graphics processing unit, an artificial intelligence accelerator, a field-programmable gate array, and an application-specific integrated circuit.

16. An apparatus for selecting a molecule, the apparatus comprising memory circuitry, machine-readable instructions, and processor circuitry to execute the machine-readable instructions to:
select one or more molecules from a data structure based on a desired olfactory profile, with the data structure being generated by an apparatus according to claim 13; and
provide information on the one or more molecules for the purpose of selecting the one or more molecules for use in one of a perfume, perfume component for another substance, cosmetic substance, and food item.

17. An apparatus for training machine-learning models, the apparatus comprising memory circuitry, machine-readable instructions, and processor circuitry to execute the machine-readable instructions to:
obtain training data, the training data comprising information on a plurality of molecules and associated olfactory profiles of the plurality of molecules;
train at least a component of at least one first machine-learning model, at least a component of at least one second machine-learning model, and a third machine-learning model using the training data,
wherein the at least one first machine-learning model is trained to output a first predicted olfactory profile of a molecule based on a first representation of the molecule,
the at least one second machine-learning model is trained to output a second predicted olfactory profile of the molecule based on a second representation of the molecule, and
the third machine-learning model is trained to output a third predicted olfactory profile of the molecule using the first predicted olfactory profile and the second predicted olfactory profile, or a combined version of the first predicted olfactory profile and the second predicted olfactory profile, as input.

18. The apparatus according to claim 17, wherein at least a component of the at least one first machine-learning model, at least a component of the at least one second machine-learning model, and the third machine-learning model are trained using supervised learning.

19. The apparatus according to claim 17, wherein at least the third predicted olfactory profile represents a plurality of olfactory labels, wherein at least a component of the at least one first machine-learning model is trained to predict a first subset of the plurality of olfactory labels and at least a component of the at least one second machine-learning model is trained to predict a second subset of the plurality of olfactory labels.

20. The apparatus according to claim 17, wherein the at least one first machine-learning model and the at least one first machine-learning model each comprise a pre-trained machine-learning model to generate an embedding of the molecule and a predictor machine-learning model to predict the respective first and second predicted olfactory profile based on the respective embedding of the molecule, wherein the third machine-learning model and the predictor machine-learning models are trained using the training data.
